(19) United States
(12) Patent Application Publication
(10) Pub. No.: US 2024/0407771 A1
(43) Pub. Date: Dec. 12, 2024

(54) ELECTROACTIVE POLYMER ACTUATOR AND METHOD OF ACTUATION THEREOF

(71) Applicant: Vicora, Inc., Buffalo, NY (US)

(72) Inventors: Daniel Rathbone, Buffalo, NY (US); Erin Garcia, Buffalo, NY (US); Richard Ducharme, Buffalo, NY (US)

(21) Appl. No.: 18/638,401

(22) Filed: Apr. 17, 2024

Related U.S. Application Data
(60) Provisional application No. 63/459,955, filed on Apr. 17, 2023.

(51) Int. Cl. A61B 17/00

(52) U.S. Cl. CPC A61B 17/00; A61B 2017/00398; A61B 2017/00871

(57) ABSTRACT

An instrument suitable for use in a surgical procedure may include (a) a body having a distal end and a proximal end, the body having a conduit between the distal end and the proximal end through which a fluid is allowed to flow under the suction; and (b) one or more actuators attached or embedded in the body, each actuator comprising one or more electroactive polymer layers that provides a mechanical motion at the distal end in response to simulation by an associated one of the electrical control signals, wherein the electrical control signals are provided in a predetermined coordinated pattern. The predetermined coordinated pattern may involve two or more groups of the actuators being activated sequentially, concurrently or a combination thereof.

### CROSS REFERENCE TO RELATED APPLICATIONS

The present application is related and claims priority to U.S. provisional application (“Parent Provisional Application”), Ser. No. 63/459,955, entitled “ACTUATED THROMBECTOMY DEVICE AND METHOD,” filed on Apr. 17, 2023.

The present application is also related to patent application of U.S. patent application (“Related Application”), Ser. No. 17/510,194, entitled “Actuated Thrombectomy Device,” filed on Oct. 25, 2021, which claims priority of (i) U.S. provisional application (“Provisional Application I”), Ser. No. 63/105,001, entitled “Actuated Thrombectomy Device,” filed on Oct. 23, 2020, and (ii) U.S. provisional application (“Provisional Application II”), Ser. No. 63/123,080, entitled “Actuated Thrombectomy Device,” filed on Dec. 9, 2020.

The disclosures of the Parent Provisional Application, Provisional Application I and Provisional Application II (collectively, “Provisional Applications”) are hereby incorporated by reference in their entireties.

### FIELD OF THE INVENTION

The present invention relates to medical devices enabled by electroactive polymers (“EAP”; e.g., piezoelectric polymers). In particular, the present invention relates to surgical instruments that includes EAP-based actuators or other mechanical components capable of axial, radial, torsional or helical motion.

### SUMMARY

According to one embodiment of the present invention, an instrument used in a medical procedure, the instrument being electrically connected to a controller that provides electrical control signals and controls mechanical suction, the instrument includes (a) a body having a distal end and a proximal end, the body having a conduit between the distal end and the proximal end through which a fluid is allowed to flow under the suction; and (b) one or more actuators attached or embedded in the body, each actuator providing a mechanical motion at the distal end in response to stimulation by the electrical control signals. The actuators may be EAP-based. The stimulation may be electrical control signals of a predetermined coordinated pattern. The predetermined coordinated pattern may involve two or more groups of the actuators being activated sequentially, concurrently or a combination thereof. For example, in one embodiment, the predetermined coordinated pattern involves two or more groups of the actuators being activated sequentially, one group after another, and wherein, within each group, two or more actuators are actuated concurrently. In one embodiment, each actuator extends along a longitudinal direction of the body and at least two or more groups of actuators are each located at a substantially equal radial distance from a longitudinal axis of the conduit.

According to one embodiment of the present invention, activating the mechanical motions of the actuators are coordinated with the pressure of the suction. For example, the suction pressure may be decreased when one of the mechanical motions causes a forward motion at the distal end of the body.

According to one embodiment of the present invention, the distal end of the body of the aspiration instrument has an opening that exposes the conduit, and wherein the portion of the body at the opening has the shape of a funnel.

According to one embodiment of the present invention, a first one of the actuators of the aspiration instrument wraps around the body helically. In another embodiment, a second one of one of the actuators also wraps around the body helically, but in a different chirality as the first actuator.

According to one embodiment of the present invention, a first one of the actuators of the instrument wraps substantially circumferential around the body relative to a longitudinal axis of the body. The two ends of the actuator may abut each other during that actuator's mechanical motion. Alternatively, the two ends of the actuator may be separated from each other by a gap and wherein the actuator includes a stiffening material that spans the gap.

According to one embodiment of the present invention, the aspiration catheter includes a mass placed on one of the actuators to modify a resonant frequency of the mechanical motion of that actuator. The mass may include a radiopaque material that can be used to guide steering of the catheter. The mass may also include tungsten to provide a tunable effect on the mechanical motion of the actuator, such as its resonant frequency.

According to one embodiment of the present invention, each actuator of the instrument may include one or more portions that are stiffened to constrain the mechanical motion of that actuator. The stiffened portions of the body may be provided by a high-modulus material or a compliant material, high-modulus or compliance being relative to the EAP layers in the actuator or the material serving as a substrate.

According to one embodiment of the present invention, the body may include a patterned or textured layer of material exposed to the conduit. The patterned or textured layer of material may be formed out of polyvinylidene fluoride (PVDF). The patterned or textured layer may include fluted or raised portions, wherein adjacent fluted or raised portions are separated from each other by a channel. In that embodiment, each raised portion may include a smooth surface or a textured surface. Alternatively, the patterned or textured layer may include etched depressions. Such etched depression may be provided in the form of a rifled helical pattern extending longitudinally along the body. The patterned or textured layer may also be formed in a helical screw pattern that extends longitudinally along the body.

According to one embodiment of the present invention, one or more sensors may be provided in conjunction with the EAP actuator. For example, one or more portions of the patterned or textual layer (e.g., at the distal end) may serve as an electroactive sensor. The sensor may be located anywhere within the catheter body, or external to the catheter body (e.g., in a tubing attached to the catheter). The sensor may be used to detect the presence of a blood clot and to provide an electrical signal as sensory output. The sensory output of the electroactive sensor, or other sensors may be fed back to the controller to modulate pressure of the suction, the frequency or amplitude of an EAP actuator's vibration, or any combination thereof.

According to one embodiment of the present invention, the patterned or textured layer may incorporate a mobile element capable of axial motion in such a way as to urge an ingested blood clot to a proximal motion. In one embodiment, the patterned or textured layer includes flexure beams that constrain an actuator to effectuate axial motion primarily. In one embodiment, the patterned and textured layer further includes a compliant region. One implementation of the compliant region includes flexure beams interconnected elongation elements. In another implementation of the compliant region, an EAP actuator is provided attached to a region of material having a lower modulus than a higher modulus region.

Various embodiments of the present invention each also include a mechanical structure at the opening to the aspiration conduit that provides a soft flared-opening to the aspiration structure when in an open position, but that is designed to have a strict closure limit which prevents a collapse of the opening to the aspiration conduit.

The present invention is better understood upon consideration of the detailed description below in conjunction with the accompanying drawings.

### BRIEF DESCRIPTION OF THE DRAWINGS

FIG. 1A is a top view showing, at the distal end of instrument 100, a cross section through distal tip 101 and catheter shaft 104, according to one embodiment of the present invention.

FIG. 1B is a cross-section, transverse to the cross-section of FIG. 1A, of distal tip 101, showing electrode layers 108 and EAP layers 109.

FIGS. 2A-1 and 2A-2 show, respectively, a side view and a transverse cross section of distal tip 200 that includes multiple individually controlled actuators (e.g., actuators 201a-201c), according to one embodiment of the present invention; in FIGS. 2A-1 and 2A-2, the actuators are in their relaxed states.

FIG. 2A-3 shows, when activated, the actuators provide a circumferential bending (i.e., radial) of compliant layer 203, which is concentrated by the action of substrate layer 202.

FIGS. 2A-4(i) to 2A-4(iii) provide examples of radial bending.

FIGS. 2A-5(i) and 2A-5(ii) illustrate a torsional response.

FIGS. 2A-6(i) and 2A-6(ii) illustrate an axial response.

FIG. 2A-7 illustrates a helical response (i.e., a combination of a torsional response and an axial response).

FIGS. 2A-8(i) and 2A-8(ii) illustrate the simultaneous or unison response in distal tip 200 of FIG. 2A-1.

FIGS. 2A-9(i) and 2A-9(ii) illustrate the grouped or coordinated response in distal tip 200 of FIG. 2A-1.

FIGS. 2B to 2D each illustrate one of three activation patterns (sequentially, simultaneously, or grouped); under each activation pattern, four activation control signals—respectively labeled 201a-201d—correspond to the control signals sent to actuators 201a-201b.

FIG. 2E illustrates coordinating an activation of an EAP actuator with an aspiration pattern, according to one embodiment of the present invention.

FIG. 3A-1 shows funnel-shaped distal tip 300 that includes actuators (e.g., actuators 301a-301c) provided around the distal tip of instrument 300, according to one embodiment of the present invention.

FIGS. 3A-2 to 3A-4 illustrate another construction for a funnel-shaped distal tip, according to one embodiment of the present invention.

FIG. 3B shows helically wrapped actuator 321 around distal tip 300, according to one embodiment of the present invention.

FIG. 3C shows high modulus material 322 being helically counter-wrapped (i.e., in an opposite chirality) against helical actuator 321 on distal tip 300, in accordance with one embodiment of the present invention.

FIG. 3C shows tubular instrument 350 with EAP actuator 326 circumferentially wrapped around the body of tubular instrument 150 between tip 300 and proximal portion 305, according to a second embodiment of the present invention.

FIG. 3D shows distal tip 300 of tubular instrument 350 with EAP actuator 326 circumferentially wrapped around the body of tubular instrument 350 between a distal portion of distal tip 300 and a proximal portion 305 of distal tip 300, according to a second embodiment of the present invention.

FIG. 3E shows tubular instrument 370 having circumferentially wrapped EAP actuators 326-1, 326-2, 326-3 and 326-4, according to one embodiment of the present invention.

FIG. 3F shows respectively mass 345 being placed longitudinally at a distal end of distal tip 300, in accordance with one embodiment of the present invention.

FIG. 3G shows, for example, multiple stiffened actuators, including actuators 341-1 and 341-2 being integrated in the configuration of FIG. 2A, in accordance with one embodiment.

FIG. 3H-1 to 3H-3 each show a configuration whereby motion in actuator 360 is enhanced by its substrate 351, according to one embodiment of the present invention.

FIGS. 31-1 and 31-3 illustrate a passive action in distal tip 300 of instrument 380, according to one embodiment of the present invention.

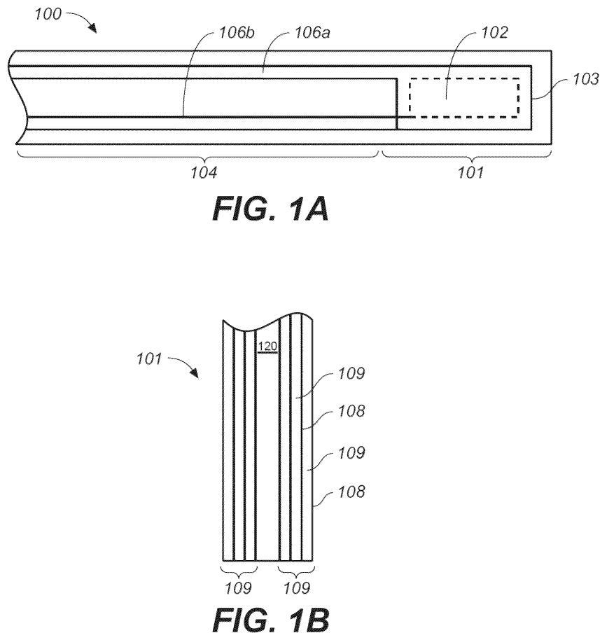

FIGS. 3J-1 to 3J-4 illustrate incorporating a second passive mechanical action to distal tip 300, according to another embodiment of the present invention.

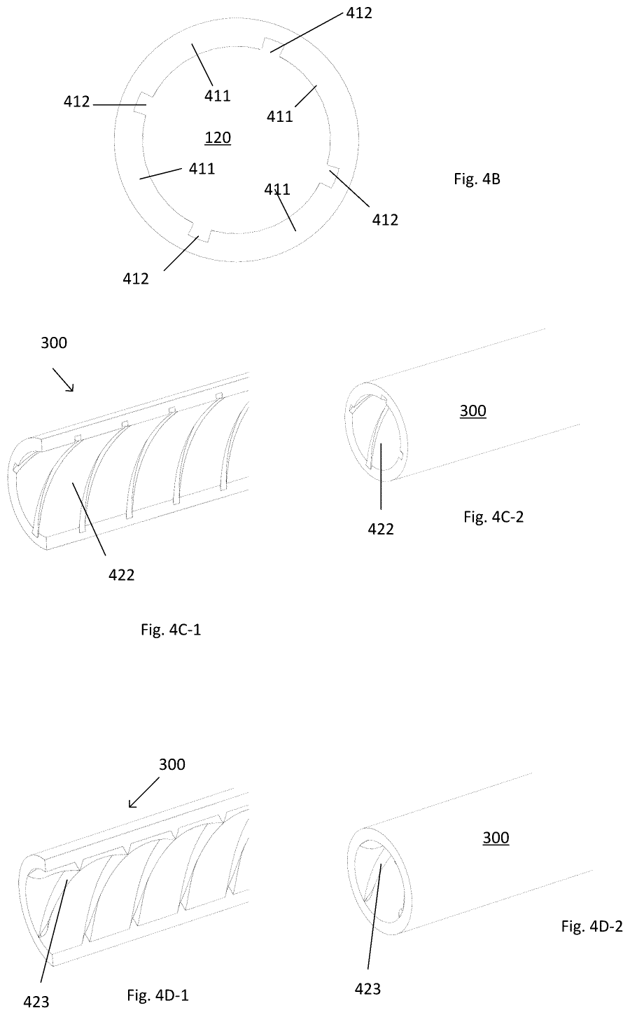

FIG. 4A-1 shows textured surface 410 provided on the walls of lumen 120 in distal tip 300 of a catheter, according to one embodiment of the present invention.

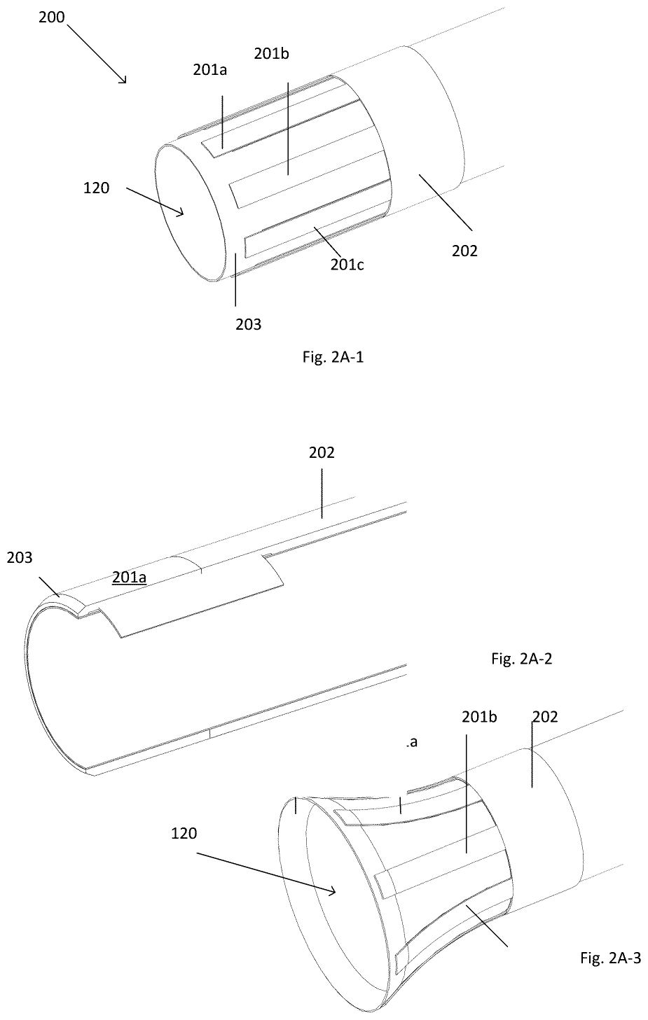

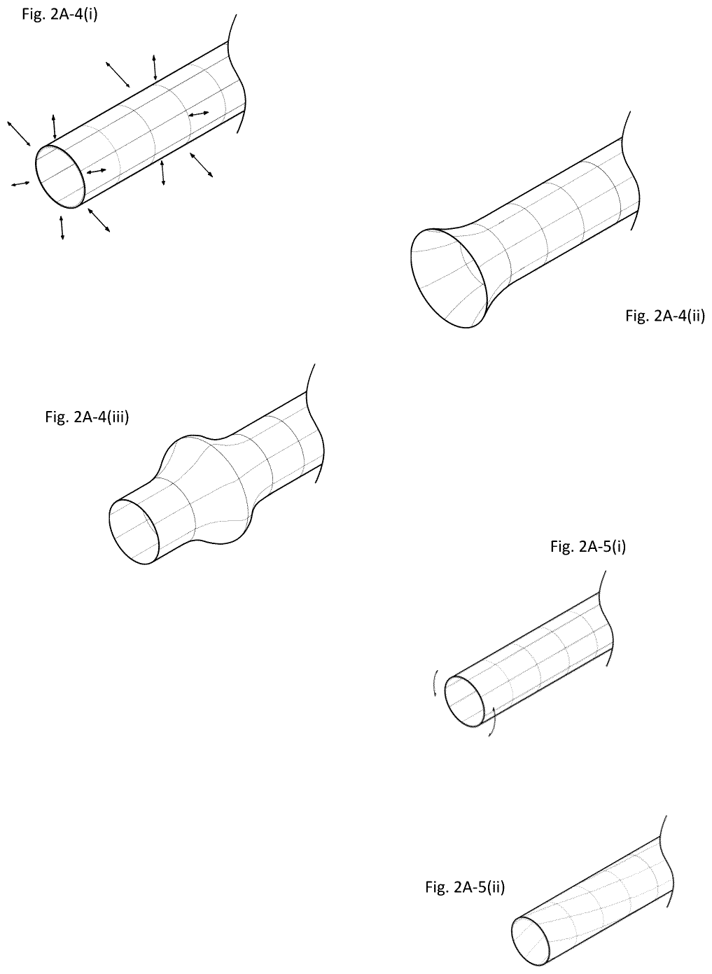

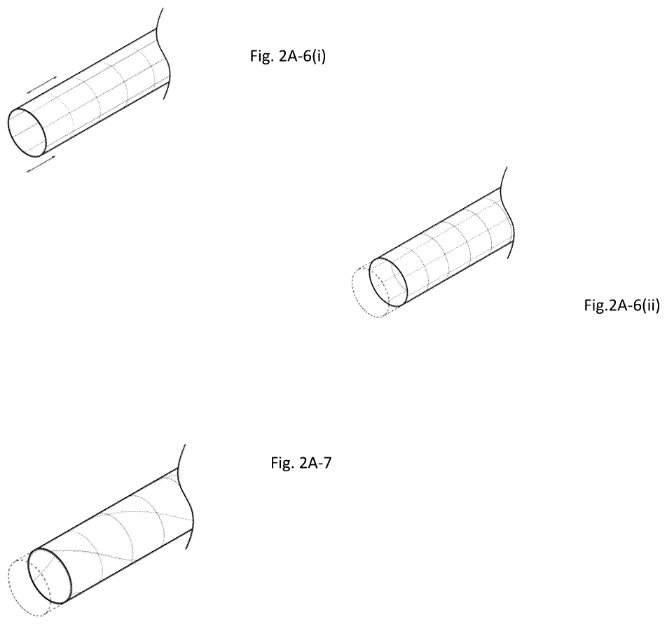

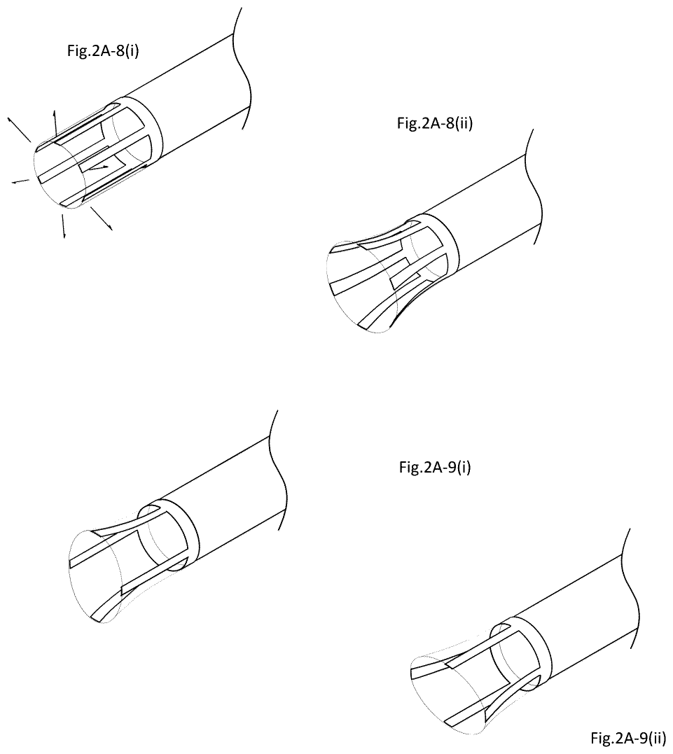

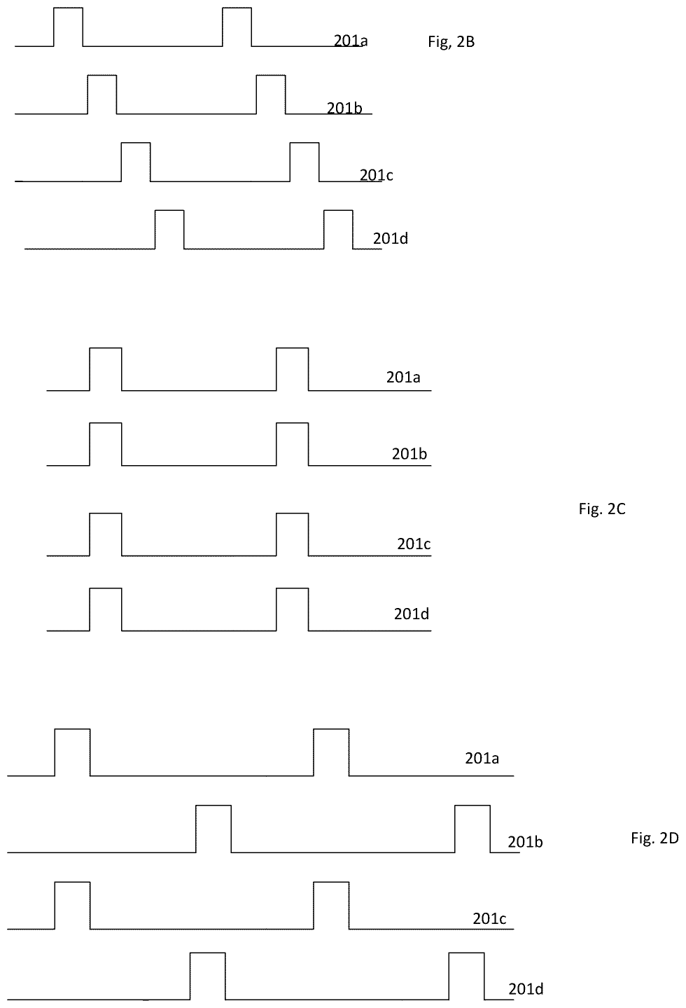

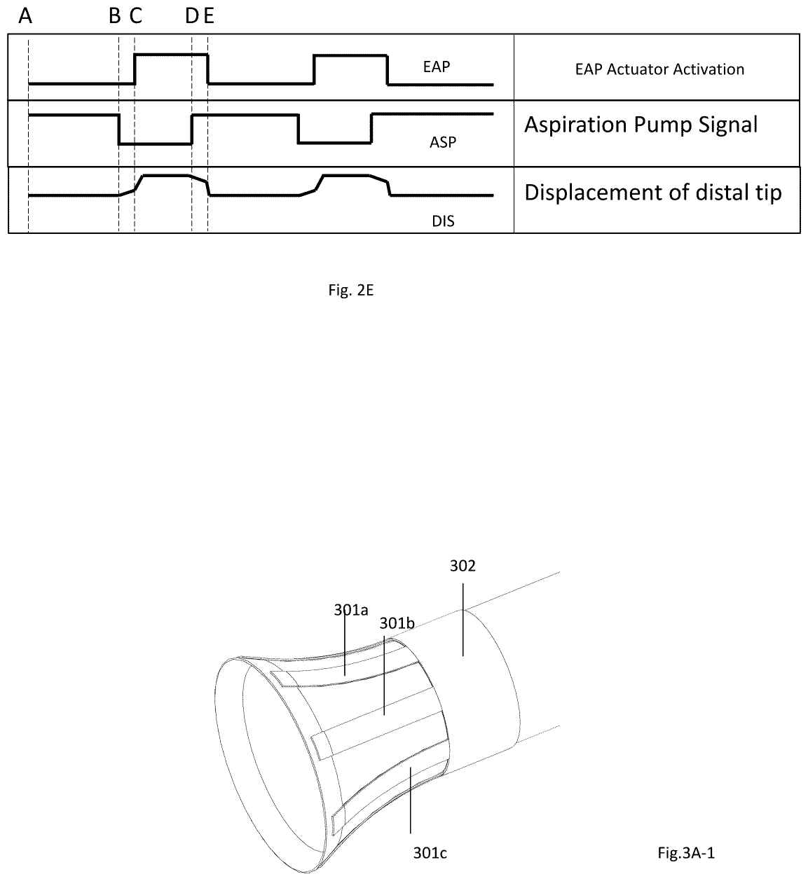

FIGS. 4A-2 and 4A-3 show side and perspective views of exemplary textured surface 410, respectively, provided as steps cut in the inside wall of lumen 120.

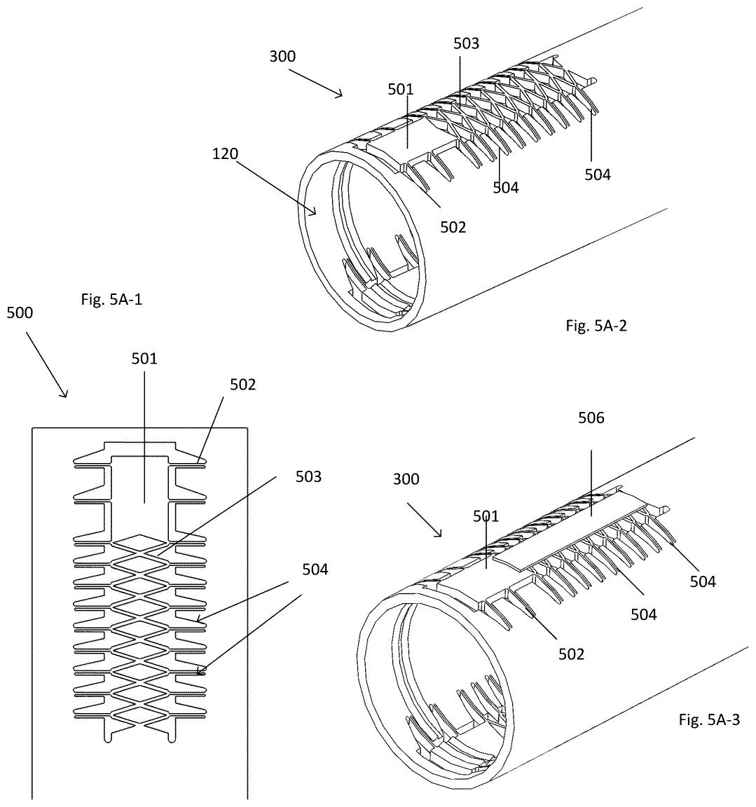

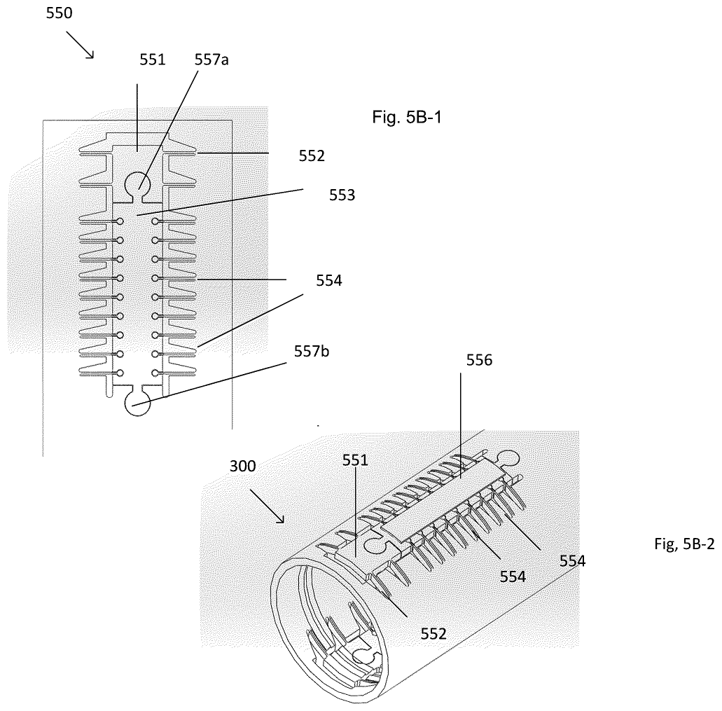

FIGS. 4A-4 and 4A-5 show transverse cross-sectional views of textured surface 410, provided in the form of patterned stepped protrusions, as viewed into the opening of lumen 120 and as viewed out to the opening of lumen 120 from inside the distal tip, respectively.

FIGS. 4A-6 to 4A-8 show (a) side and perspective transverse cross-sectional views, and (b) a perspective view of textured surface 410, provided in the form of another patterned stepped protrusions.

FIG. 4B shows the wall of lumen 120 having raised portions 411 separated by channels 412, according to one embodiment of the present invention.

FIGS. 4C-1 and 4C-2 show a transverse cross-sectional view and a perspective view of helical grooves 422 on the wall of lumen 120.

FIGS. 4D-1 and 4D-2 show a transverse cross-sectional view and a perspective view, respectively, of helical screw-like raised structure 423 on the enclosing wall of lumen 120, according to one embodiment of the present invention.

FIGS. 5A-1 to 5A-3 illustrate the operation of textured structure 500 provided on the enclosing sidewalls of lumen 120, according to one embodiment of the present invention.

FIGS. 5B-1 to 5B-2 illustrate the operation of second textured structure 550 provided on the sidewalls of lumen 120, according to one embodiment of the present invention.

FIGS. 6A-1 and 6A-2 illustrate clamshell structure 610 for controlling the opening to lumen 120, according to one embodiment of the present invention; clamshell structure 610 has a soft-opening and a stiff closure limit.

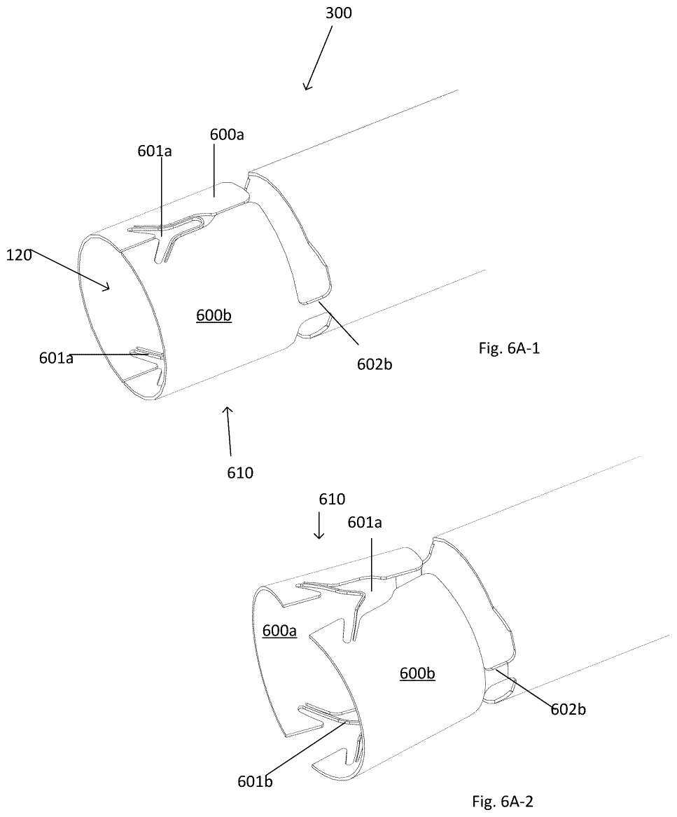

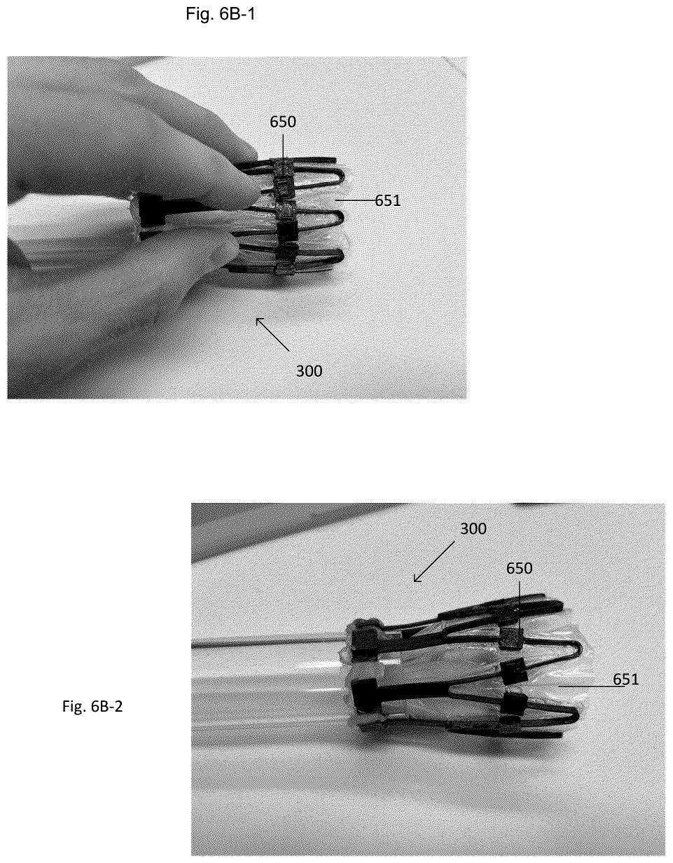

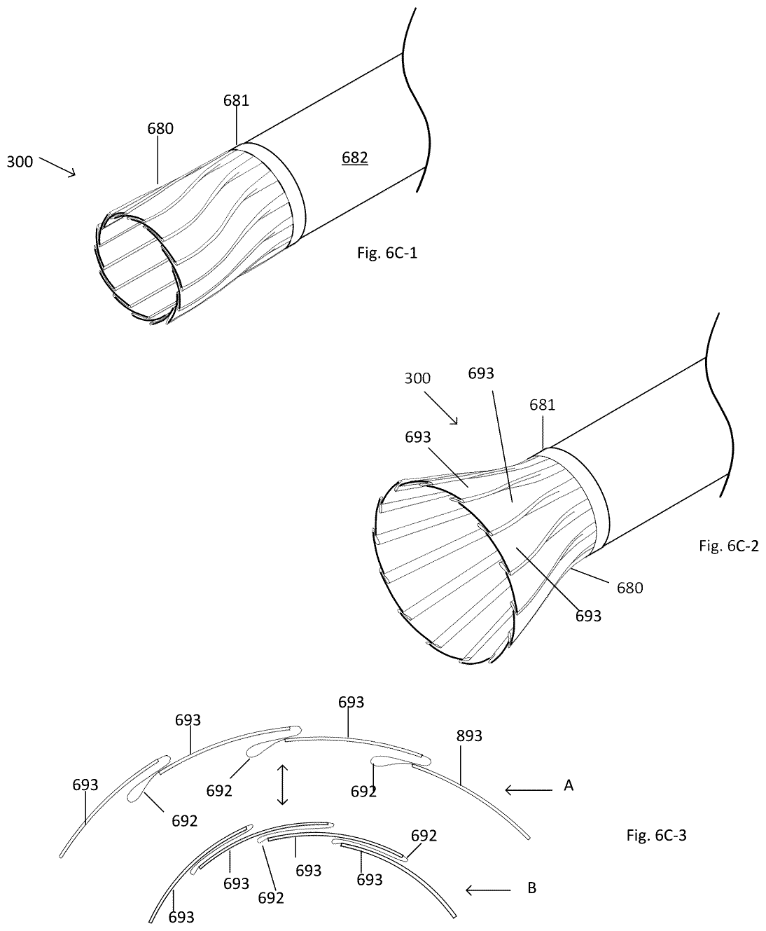

FIGS. 6B-1 and 6B2 illustrate stent-like mechanical structure 650 for controlling the opening to lumen 120, according to one embodiment of the present invention; like clamshell structure 610 of FIGS. 6A-1 and 6A-2, stent-like mechanical structure 650 also allows a soft opening, but preserves a stiff closure limit, for the opening to lumen 120.

FIGS. 6C-1 to 6C-3 illustrate the operation of tubular compliant structure 680 for controlling lumen 120 using a foldable membrane material, according to one embodiment of the present invention.

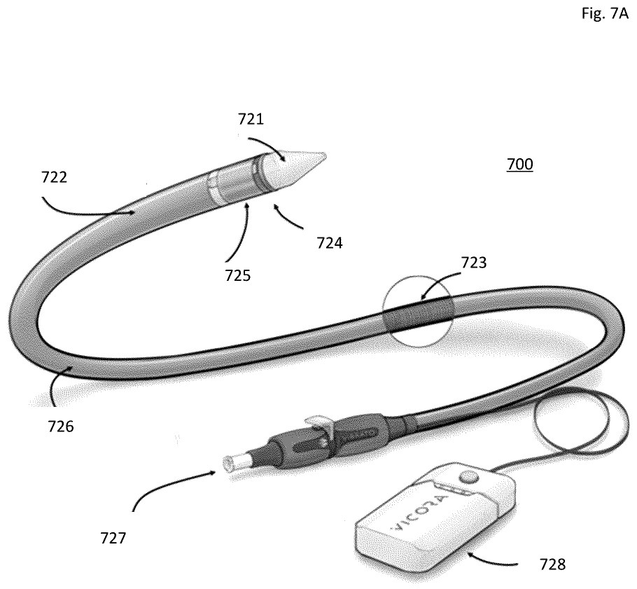

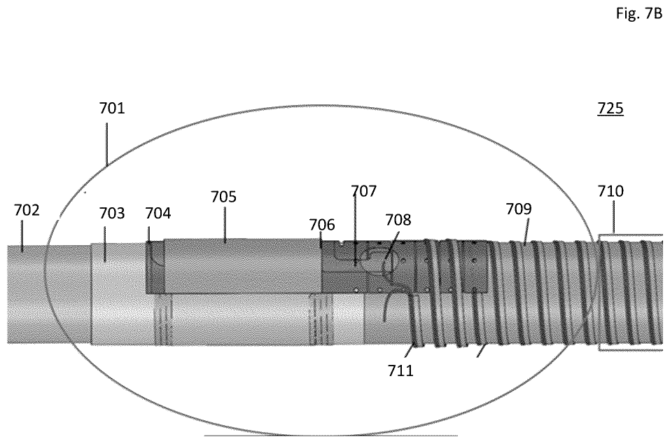

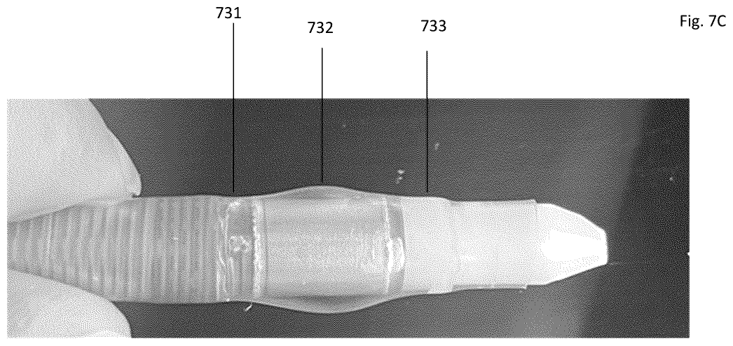

FIG. 7A shows integrated thrombectomy device 700, in accordance with one embodiment of the present invention.

FIG. 7B is an enlarged view of integrated EAP actuator 725 at distal tip 724 of integrated thrombectomy device 700, in accordance with one embodiment of the present invention.

FIG. 7C illustrates formation of the Tecoflex encapsulation in integrated EAP actuator 725, in accordance with one embodiment of the present invention.

### DETAILED DESCRIPTION OF THE PREFERRED EMBODIMENTS

The present application discloses various embodiments of an aspiration catheter or another surgical device that includes a tip at the distal end, which may be actuated to vibrate vigorously to achieve desirable mechanical effects suitable for use in various surgical applications (e.g., thrombectomy). In thrombectomy, for example, the vibration may impart a desirable force on a blood clot, thereby achieving agitation, disruption, fragmentation, compression, or breaking up of the blood clot to facilitate removal. Breaking up a blood clot, for example, is desirable to prevent “corking,” thus allowing it to be directly aspirated into the catheter or surgical instrument. Unlike devices in minimally invasive surgery, where the tissues subject to the procedure are accessed through conveniently located small incisions, a location in a blood vessel is typically accessed through a long flexible catheter, often 100 cm or more in length. However, while this detailed description provides examples of implementing the present invention in conjunction with a catheter, the present invention may be implemented even in minimally invasive surgical devices as well. Thus, in this detailed description, the term “instrument” should be taken to mean a device associated with a catheter or any suitable surgical device.

One or more electroactive polymer (EAP) actuators may be provided at the tip in the distal end (“distal tip”) of the instrument. Each EAP actuator may be actuated (e.g., set in motion or vibration) by electrical signals transmitted from the proximal end of the instrument. In this arrangement, the mechanical motion of the distal tip may be confined and not transferred to any substantial length of the instrument. In some embodiments, the actions of the EAP actuators need not be linear. The linear motion may be supplemented by other mechanical motion of the distal tip. For example, in some embodiments the range of mechanical motions at the distal tip may include any radial, axial, torsional and helical motion, or combination thereof. In this context, a torsional motion refers to a rotational motion about the axial direction and a helical motion refers to a combination of torsional and axial motions. Suitable electroactive polymers include various combinations of vinylidene fluoride (VDF), trifluoroethylene (TrFE), 1,1-chlorofluoroethylene (CFE), and chlorotrifluoroethylene (CTFE). For example, the terpolymers P (VDF-TrFE-CTFE) and P (VDF-TrFE-CFE) are available commercially from Piezotech (a subsidiary of Arkema S.A., Paris, France). These terpolymers, which have different electroactive properties, exhibit large electrostrictive strain (e g., greater than 0.5%, preferably greater than 3.0%) under electric fields of 20-200 V/μm (e.g., 20-100V/μm; preferably, about 50V/μm).

FIG. JA is a top view showing distal tip 101 at the distal end of instrument 100, and instrument shaft 104, according to one embodiment of the present invention Distal tip 101 may be itself an actuator or includes one or more actuators that are each capable of electrically controlled motion. Instrument 100 includes a proximal end 105 (not shown) with a watertight connection to an electronic drive circuit to receive an electrical signal (e g., 20-200 Hz; preferably, 50-150 Hz), optimized to a resonant frequency of distal tip 101, so that the instrument is suitable for both fracturing a blood clot and ingesting the debris of the blood clot by aspiration (“suction”). Better blood clot ingestion performance is believed favored at the higher end of the frequency range. For the intended operations, each electrical signal may have, for example, amplitudes of 50.0-300.0 volts, with or without a DC offset

Instrument shaft 104 may be of a conventional mechanical design, such as having an inner layer or liner of poly-tetrafluroethylene (PTFE), Pebax or thermoplastic polyurethane (TPU). The PTFE inner layer may be surrounded by an outer layer of a reflowable material (e.g., Pebax with varying durometers across the length of instrument shaft 104). In addition, instrument shaft 104 accommodates both active electrode 106a and return electrode 1066, which are electrically insulated from each other, each electrode extending along the entire length of instrument shaft 104. These electrodes may be formed out of any suitable electrically conductive wires. The inner layer or the conductive wires may be provided with suitable mechanical strength, or in the form of a braid or coil, so as to provide instrument shaft 104 mechanical integrity and kink resistance The conductive wires may be embedded in an electrically non-conductive braid or a coil (e.g., constructed from poly-ether-ether ketone (PEEK)) that extends along the entire length of instrument 100. These braids or coils are available in various patterns from, for example, Steeger USA, US Biodesign, Inc., and Admedes, Inc. Alternatively, an all-metallic braid or coil with electrically insulated wires for active electrode 106a and return electrode 1066 are also possible. However, embedding the electrodes in a non-conductive braid or coil is preferable to avoid shorting. Although, purely for illustrative purpose, only active electrode 106a and return electrode 106b are shown in FIG. 1A, any suitable number of active electrodes and return electrodes may be used.

Distal tip 101 at the distal end of instrument 100 is configured for engaging a thrombus. Distal tip 101 has preferably a flush or angled tip, so as to take maximal advantage of an opening through which the blood clot may be ingested. Layers of the EAP are embedded inside distal tip 101. Each EAP layer strains when an electric field is placed across it. (Note that, although a greater strain is achieved at a greater electric field, the strain-electric field relationship is generally non-linear.) As shown in FIG. 1A, the EAP layers are each provided between thin and flexible layers of electrodes, e.g., between electrode 102 and electrode 103, which is underneath electrode 102. Electrodes 102 and 103 are each electrically connected to either active electrode 106a or return electrode 1066. In this manner, movement occurs only at distal tip 101 at the distal end of instrument 100 and no energy is lost in moving active electrode 106a and return electrode 1066 in instrument shaft 104 In one embodiment, each EAP layer may be between 2-20 μm thick. Distal tip 100 may move in both the longitudinal direction and in the transverse direction.

According to one embodiment of the present invention, each EAP layer may be formed by dip-coating. For example, distal tip 101 at the distal end of instrument 100 may be dipped in a solution of the EAP in a polar solvent, such as diethyl formamide (DMF) or methyl ethyl ketone (MEK). In this manner, coaxial 20-200 μm thick EAP layers may be formed in distal tip 101 in successive dips. After forming each EAP layer, an electrode layer is formed over the exposed surface of the EAP layer by, for example, sputtering (e.g., gold or aluminum), clip-coating (e.g., silver-embedded urethane), pad printing or spray coating using a conductive electric ink or a particle-free metal-complex conductive ink (e.g. conductive inks available from Electroninks or LiquidX). The forming steps for the EAP layer-electrode layer combination may be repeated multiple times. The electrode layers thus formed may be connected to either active electrode 106a or return electrode 106b, such that electrodes of opposite polarities are formed on opposite sides of an EAP layer, creating in effect a capacitor. FIG. 1B is a cross-section, transverse to the cross-section of FIG. 1A, of distal tip 101, showing electrode layers 108 and EAP layers 109. Depending on the mechanical properties desired, each EAP layer may have any one of various thicknesses. Additional non-EAP layers (not shown) may also be included.

Fragmented or compressed blood clot resulting from the motions of the distal tip of instrument 100 may be removed from the blood vessel using aspiration. Clot ingestion efficiency depends on a number of factors (e.g., the actuator's activation parameters, in conjunction with any cyclic or variable aspiration patterns). The inventors believe that the motions of the distal tip may compress a blood clot. Such compression may remove serum from the blood clot, thereby reducing the volume of the blood clot and thus facilitates ingestion of the blood clot.

An aspiration pattern is created by turning on and turning off a suction mechanism according to a predetermined wave pattern. Prior art aspiration pressure patterns use very low frequency changes (e.g., 6-12 Hz), as the pressure pattern is driven from the proximal end of instrument 100. When EAP actuators are integrated into an instrument, the inventors have discovered that very strong suction may create a force that impedes the outward component of the longitudinal or axial motion of distal tip 100. According to one embodiment of the present invention, the aspiration patterns and the vibration patterns are electronically controlled in a cooperative manner to optimize both instrument tip movement and clot ingestion. For example, when the longitudinal component of distal tip 100's motion is outward, suction is reduced. Conversely, when the longitudinal component of distal tip 100's motion is inward, suction is increased. With distal tip motion and aspiration pressure coordinated, a much faster response time may be achieved and a wider frequency response range—up to 1 KHz—may be utilized.

Lumen 120, which runs substantially the entire length of the shaft in instrument 100, provides a conduit for the aspiration intake and discharge. Polyvinylidene fluoride (PVDF) co-polymer sensors may be positioned inside lumen 120 at its distal end and elsewhere to detect when instrument 100 has become “corked” or plugged. A suitable tactile pressure sensor may be, for example, any of the pressure-sensing guidewires disclosed in U.S. patent application Ser. No. 17/510,257, entitled “Pressure-Sensing Guidewire,” filed on Oct. 25, 2021. Sensing the clogged or plugged condition triggers causes the controller to change its aspiration and vibration patterns to “unplug” the instrument.

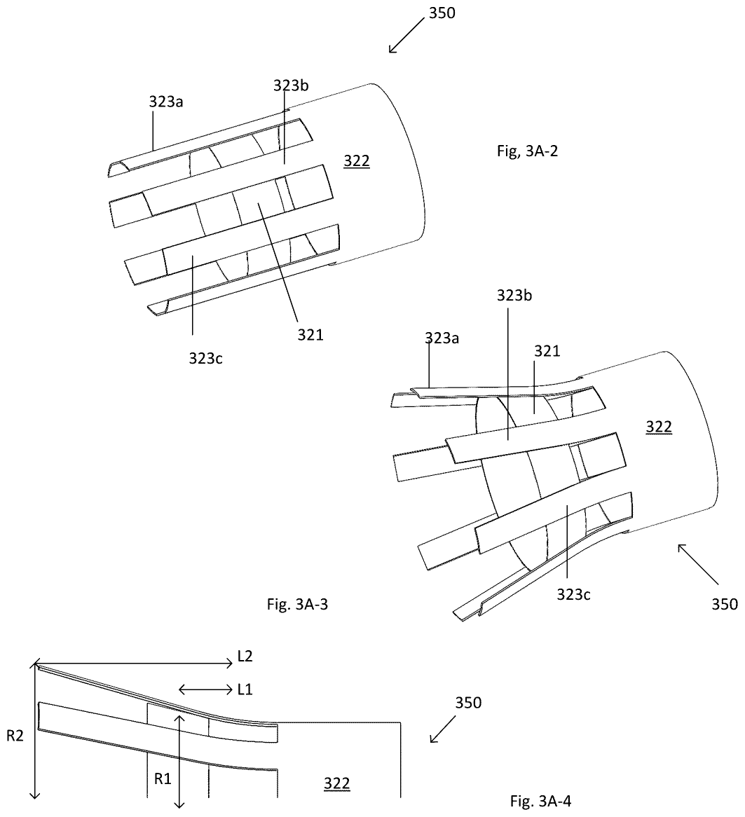

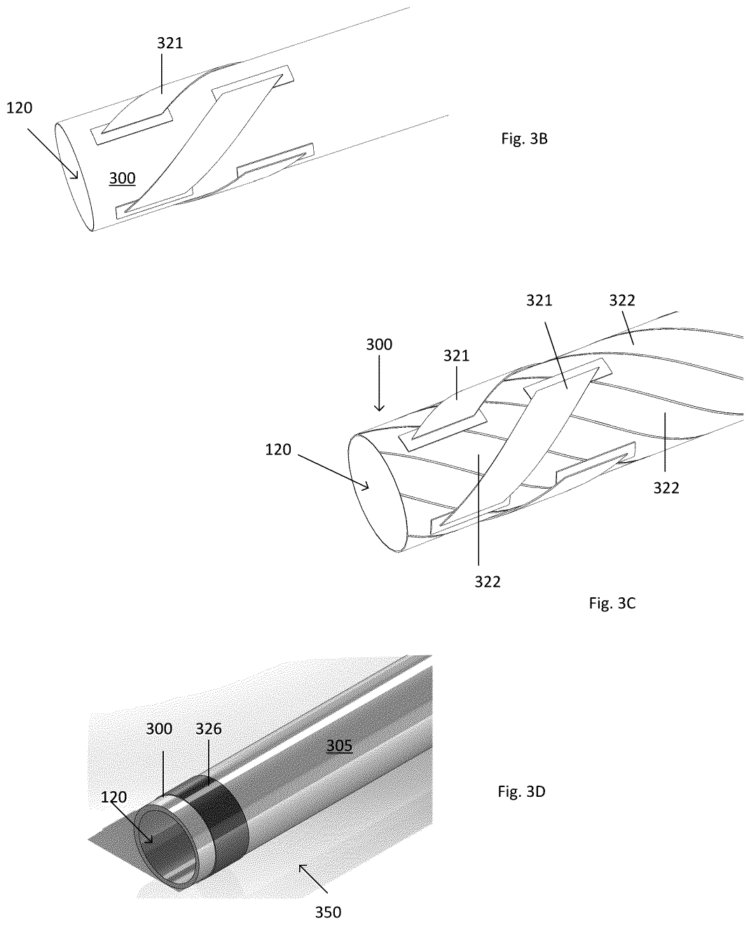

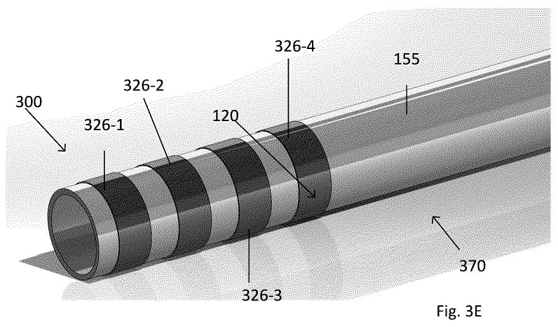

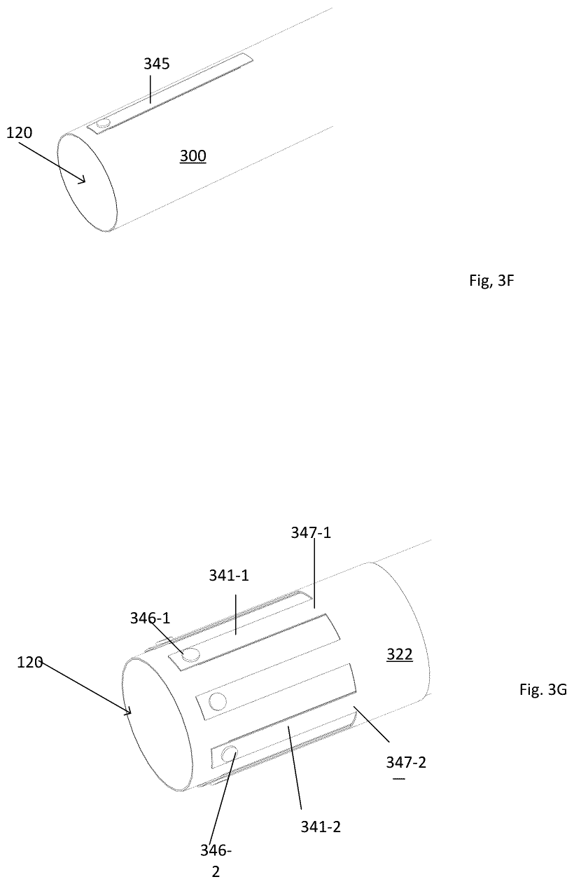

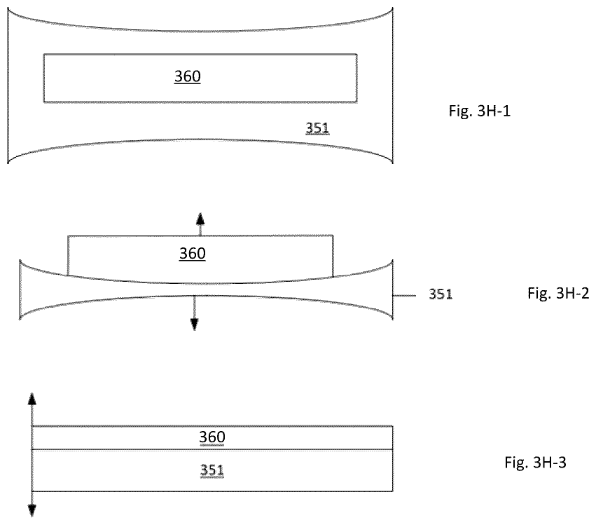

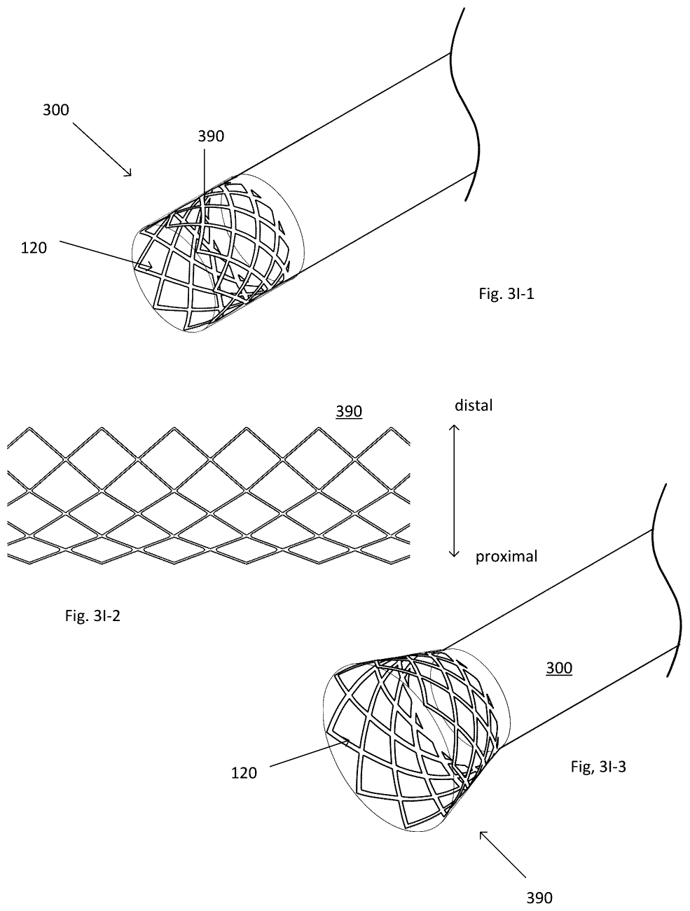

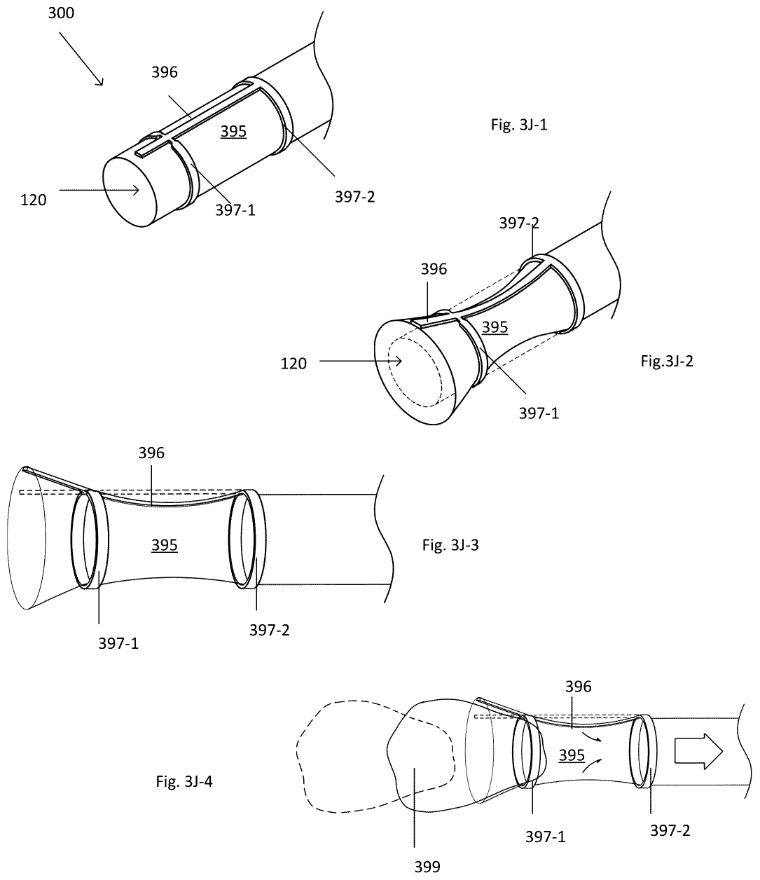

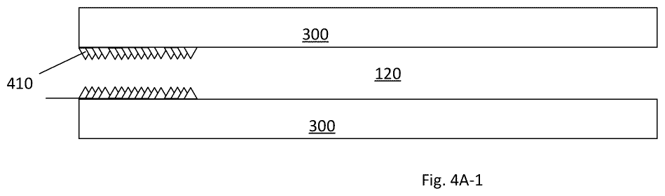

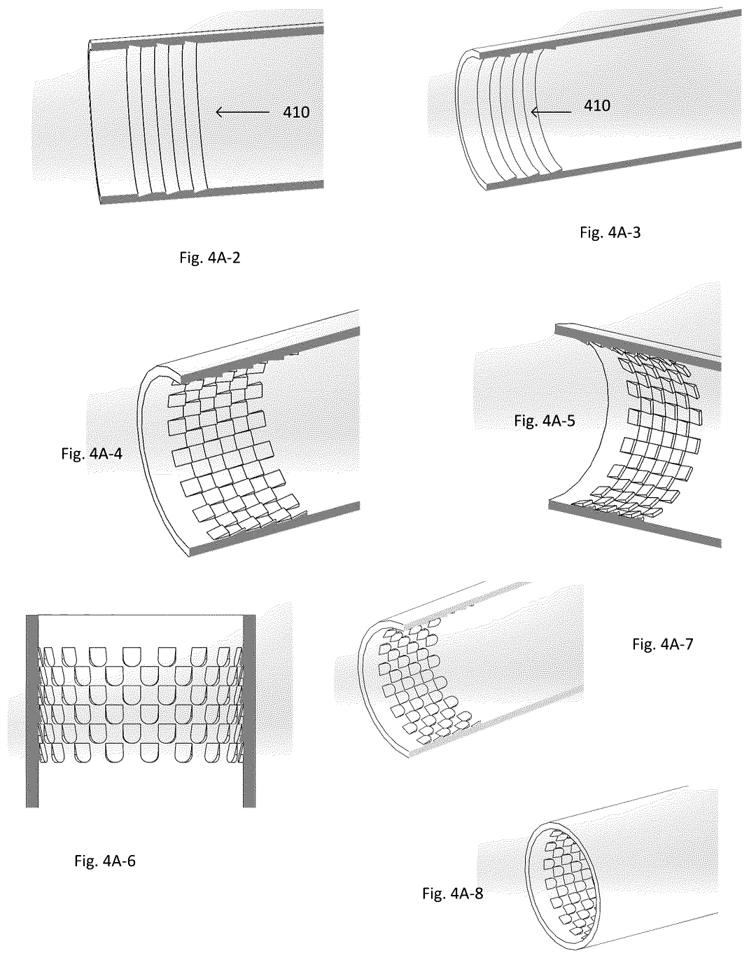

In the embodiments described above, the electrodes to the EAP layer or layers of the actuators are individually provided. Multiple actuators can be integrated into the tip of the instrument as straight sections and actuated independently. For example, FIGS. 2A-1 and 2A-2 show, respectively, a side view and a transverse cross-sectional view of distal tip 200 that includes multiple individually controlled actuators (e.g., actuators 201a-201d; actuator 201d situated on opposite side and thus not seen in FIG. 2A-1), according to one embodiment of the present invention. In this configuration, each actuator may be an axially or longitudinally aligned cantilever beam encapsulated at its proximal end to circumferentially wrapped substrate layer 202. As shown in FIG. 2A-1, inner layer 203 of a compliant polymer material may be provided to line lumen 120. Circumferentially wrapped substrate layer 202 overlays both the proximal ends of the actuators and inner compliant layer 203. Substrate layer 202 may be formed of a high modulus material relative to the material in the immediate surrounding (e.g., compliant layer 203) 1C. FIG. 2A-1 shows the actuators in a relaxed state. When activated, each actuator extends in the axial or longitudinal direction. As circumferentially wrapped substrate layer 202 stiffens the proximal portion of distal tip 200, a preferential circumferential bending (i.e., radial) of compliant layer 203 by the actuators provides a flaring (i.e., increase in diameter) at the opening of lumen 120 at the distal tip of instrument 200, as shown in FIG. 2A-3.

Using a combination of actuation patterns, a medical professional can drive actuators 201a-201d, individually and collaboratively, to achieve various effects. For example, FIGS. 2A-4(i) to 2A-4(iii) provide examples of radial bending. FIG. 2A-4(i) illustrates radial expansions at the far end of the distal tip and a middle section of the distal tip, respectively provides a flare-out response (FIG. 2A-4(ii)) and a radial bulging response (FIG. 2A-4(iii)). Similarly, FIGS. 2A-5(i) and 2A-5(ii) illustrate a torsional response; FIGS. 2A-6(i) and 2A-6(ii) illustrate an axial response; and FIG. 2A-7 illustrate a helical response (i.e., a combination of a torsional response and an axial response).

FIGS. 2B-2D illustrate the control signals to actuators 201a-201c under a sequential activation pattern, a concurrent or unison actuation pattern, and a coordinated or grouped actuation pattern, respectively. In FIG. 2B, for example, under the sequential actuation pattern, actuators 201a-201b are each actuated according to a predetermined order. In FIG. 2C, under a concurrent or unison actuation pattern, actuators 201a-201b are actuated simultaneously. FIGS. 2A-8(i) and 2A-8(ii) illustrate the simultaneous or unison response in distal tip 200 of FIG. 2A-1. In FIG. 2D, under a coordinated actuation pattern, actuators 201a and 201c and actuators 201b and 201d form two groups that are actuated sequentially. Within each group, however, the actuators are actuated simultaneously. FIGS. 2A-9(i) and 2A-9(ii) illustrate the grouped or coordinated response in distal tip 200 of FIG. 2A-1. In the actuation pattern of FIG. 2D, actuators 201a and 201c and actuators 201b and 201d form two opposite-facing actuators groups. In embodiments having even more actuators, an even more complex but suitable actuation pattern can be constructed. Of importance, the actuators can be activated by combining actuation patterns into a sequence of actuation patterns to tailor a desirable specific result.

FIG. 2E illustrates coordinating an activation of an EAP actuator with an aspiration pattern, according to one embodiment of the present invention. As shown in FIG. 2E, at the end of the period between time points A and B, aspiration pump signal ASP deactivates (i.e., reduces) the aspiration suction slightly ahead of activation signal EAP activating one or more EAP actuators at time point C. At time point D, just slightly ahead of activation signal EAP deactivating the EAP actuators at time point E, aspiration pump signal ASP reactivates (i.e., increases) aspiration suction again. This activation pattern can be repeated (as shown in FIG. 2E) or varied in any suitable manner. In FIG. 2E, the mechanical response of the distal tip is illustrated in displacement waveform DIS.

According to one embodiment of the present invention, however, EAP actuators may be integrated with other mechanical or electrical elements into an actuator (“integrated EAP actuator”) that can be used as a building block for constructing an instrument. An integrated EAP actuator may have characterized electromechanical properties and may be formed to have any desired geometry for deployment in an instrument (e g., distal tip 101 at the distal end of instrument 100) Thus, one or more integrated EAP actuators may be incorporated into distal tip 100 (e.g., as a three-dimensional array of integrated actuators) at the distal end of instrument 100

Each of the embodiments described herein may be driven by a drive electronic circuit. If distal tip 101 is designed to have multiple independently controlled actuators, more than one waveform may be provided to each of the active electrodes. In most embodiments described above, the drive circuit may provide driving waveforms, for example, between 50.0-250.0 volts (peak-to-peak). The driving waveform may be sinusoidal, triangular, square or any desired wave shape (preferably, a square wave, such as shown in FIG. 2B-2D) to provide the greatest acceleration or vibration. A suitable driving circuit may be provided, for example, using Microchip HV56020 or Microchip HV 56022.

The shape of the distal tip may be any of various suitable shapes. For example. FIG. 3A-1 shows funnel-shaped distal tip 300 that includes actuators (e.g., actuators 301a-301c) provided around distal tip 300, according to one embodiment of the present invention. The actuators may be provided as cantilever beams, each supported at one end by higher modulus substrate 302, as illustrated above with respect to FIGS. 2A-1 to 2A-3. Having actuators 301a-301c on funnel-shaped distal tip 300 are merely illustrative. Any suitable number of actuators in any suitable placement may be used For example, the actuator arrangement of FIGS. 2A-1 to 2A-3, and their actuation patterns (e.g., as illustrated by a 4-actuator configuration in FIGS. 2B-2D) are also suitable. Furthermore, actuators 301a-301c need not act in a longitudinal direction. As explained in further details below, different configurations of actuators are possible to achieve specific desired results The funnel configuration allows for greater tip displacement and for “grooming” a clot into a preferential geometry to facilitate ingestion.

In FIG. 3A-1, the proximal end (i.e., away from the funnel opening) of each of actuators 301a-301c may be preferentially stiffened by a relatively more rigid actuator material (i.e., with a higher modulus), which limits bending at the proximal end and enables the distal end to have an increased degree of freedom and greater displacement in both the forward and traverse axial directions. In this detailed description, the “forward” or “outward” direction refers to the direction of increasing distance from the proximal end. The greater displacement is believed to be beneficial for clot ingestion.

FIGS. 3A-2 to 3A-4 illustrate another construction for a funnel-shaped distal tip, according to one embodiment of the present invention. As shown in FIG. 3A-2, rather than individually actuatable cantilever beams, distal tip 350 includes a substrate layer, which is formed into circumferential portion 322 and multiple cantilever beam portions (e.g., cantilever beam portions 323a-323c). In FIGS. 3A-2 to 3A-4, an underlying compliant layer that lines lumen 120 is omitted for clarity. The cantilever beams are provided around annular EAP actuator 321, which expands radially when activated. The substrate layer may be formed of a higher modulus material relative to, for example, the underlying compliant layer. However, each cantilever beam portion may be made flexible by performing a localized thickness removal at a pivot point. The pivot point may be provided, for example, at where the cantilever beam extends from circumferential portion 322. The localized thickness removal may be accomplished, for example, by laser ablation. FIGS. 3A-2 and 3A-3 show the relaxed and activated states of distal tip 350. In the activated state, the expansion of EAP actuator 321 flares out the cantilever beams to expand the diameter of distal tip 350.

FIG. 3A-4 illustrates the mechanical action of the expansion of EAP actuator 321. As shown in FIG. 3A-4, expanded EAP actuator 321 has an outer radius R1, which is amplified by the cantilever beams to result in a flared-out radius R2 at the opening to lumen 120. In this configuration, EAP actuator 321 is distance L1 from the pivot point, while the flared-out opening of distal tip 350 has distance L2 from the pivot point. The limited strain of EAP actuator 321 is amplified in this configuration by according to the relation

$$\frac{R 1}{R 2} = \frac{L 1}{L 2} .$$

The actuators of the present invention can also be integrated into the distal tip as one or more long strips by wrapping around the distal tip in a helical pattern that extends further in the longitudinal direction towards the proximal end of the instrument. FIG. 3B shows helically wrapped actuator 321 around distal tip 300, according to one embodiment of the present invention. In fact, desirable action may be achieved using strips wrapping around the distal tip that are passive (i.e., not capable of being actively controlled). For example, additional polymeric material—whether active or passive—may be used to wrap around distal tip 300 in the same helical direction as actuator 321 (e.g., clockwise, as viewed from distal tip 300 towards the proximal end of the instrument) or in the opposite helical direction (e.g., anti-clockwise, as viewed from distal tip 300 towards the proximal end of the instrument). FIG. 3C shows high-modulus material 322 being helically counter-wrapped (i.e., in an opposite chirality or direction) against helical actuator 321 on distal tip 300, in accordance with one embodiment of the present invention.

High-modulus material 322 may itself be an EAP actuator. In one embodiment, high-modulus material 322 may be formed using a higher modulus polymer material than a relatively compliant substrate it wraps around. Counter-wrapping (i.e., having two strips of opposite chirality, as in FIG. 3C) allows an elongation of actuator 321 to combine axial and rotational motions in distal tip 300. As shown in FIG. 3C, actuator 321 may be formed using an elastomer inner liner (e.g., PTFE, Pebax, or TPU). (Pebax belongs to a class of block copolymers made up of rigid polyamide blocks and soft polyether blocks of a range of durometers.) The elastomer inner liner may be supported by high-modulus material 322 (e.g., a higher durometer Pebax) that is coiled in an opposite chirality to actuator 321, so as to enable energy being directed away from actuator 321, thereby resulting in an axial elongation of distal tip 300.

Alternatively, an actuator of the present invention may be primarily wrapped singly—i.e., wrapped circumferentially—around a distal tip of an instrument. FIG. 3D shows distal tip 300 of tubular instrument 350 with EAP actuator 326 circumferentially wrapped around the body of tubular instrument 350 between a distal portion of distal tip 300 and a proximal portion 305 of distal tip 300, according to a second embodiment of the present invention. When activated, EAP actuator 326 expands and relaxes circumferentially around distal tip 300 of instrument 350, thereby providing a radial motion that dynamically changes the internal diameter of distal tip 300 (hence, diameter of lumen 120) under EAP actuator 326. Constructed using a higher modulus material than the underlying compliant material, EAP actuator 326 provides both mechanical support and resistance to collapse during aspiration. Single-wrapped EAP actuator 326 may be formed, in one implementation, with its two ends butting against each other when actuated. In that implementation, in its non-activated state, single-wrapped 326 may have a gap between its ends. A stiffening material may be provided over the gap to reinforce single-wrapped actuator 326 in its activated state. Alternatively, single-wrapped actuator 326 may be formed with one end of the EAP strip folded over the other end.

Additional EAP actuators may be provided along proximal portion 155 to facilitate transportation of an ingested blood clot or blood clot debris down lumen 120. FIG. 3E shows tubular instrument 370 having circumferentially wrapped EAP actuators 326-1, 326-2, 326-3 and 326-4, according to one embodiment of the present invention. In some embodiments, circumferentially wrapped actuators 326-1 to 326-3 are actuated interdependently and in a coordinated fashion to create a peristaltic movement to aid passing ingested blood clots under a predetermined activation pattern. The actuator motions may also facilitate advancing instrument 370 as it navigates to a target location within the vasculature, ureters, the gall bladder, or any other suitable target locations. Integrating a stiffening material in or around an actuator can also direct energy from the actuator motion towards an optimized movement in a preferential direction.

To summarize, integrating one or more actuators into a distal tip of an instrument provides tunable mechanical energy for optimized instrument action. To further tune its performance, the instrument may further integrate a mass to thereby increase energy output at a selected resonant frequency. In some embodiments, the integrated material may be a radiopaque material, which may serve an additional purpose of providing visibility under fluoroscopy for guiding the instrument during its navigation in the patient. Alternatively, a tungsten mass may be used. Tungsten is a common material in instrument construction and thus its associated safety, manufacturability and cost characteristics are all well-understood. FIG. 3F shows mass 345 being placed longitudinally at the distal end of distal tip 300, in accordance with one embodiment of the present invention. Alternately or in addition, one or more masses may be placed further towards the proximal end (e.g., at the middle portion of distal tip 300), The resonant frequency for maximum action in mass 345 may be optimized by adjusting the size and its center of gravity of mass 345 along the length of distal tip 300's mechanical action.

In addition, the displacement of an actuator can also be optimized by constraining its movement at one or more locations, which may result in an increased action or effectiveness at another location or locations. In some embodiments, a constraint can be achieved by either attaching or encapsulating a more rigid material at the constrained location and incorporating a less rigid material at the desired location of greater displacement. FIG. 3G shows, for example, multiple stiffened actuators, including actuators 341-1 and 341-2, being integrated into the configuration of FIG. 2A, in accordance with one embodiment. As shown in FIG. 3G, both the proximal and the distal ends in each of actuators 341-1 and 341-2 may serve as a location where its respective actuator may be constrained or made compliant to allow adjusting their displacements. For example, constraining actuator 341-1 by rigidizing proximal end 347-1 may force an increased displacement along the length of actuator 341-1. The effect is further enhanced if distal end 346-1 is also constrained. If actuator 341-1 is constrained only at proximal end 347-1, and distal end 346-1 is made relatively unconstrained or compliant, actuator 341-1 will provide greatest displacement at its distal end.

In some embodiments, the constraints in an actuator may work together with a substrate to which the actuator is embedded or attached to impart a desirable action (e.g., maximum displacement) in the distal tip of an actuator. FIG. 3H-1 to 3H-3 each show a configuration whereby motion in actuator 360 is enhanced by its substrate 351, according to one embodiment of the present invention. In FIG. 3H-1, for example, actuator 300 is embedded in substrate 351 which has a varying thickness along the longitudinal length of actuator 360. As a result, depending on the modulus of substrate 351's material, and whether constraints are placed along the length actuator 360, the location of the desirable action (e.g., maximum displacement) can be controlled. FIG. 3H-2 shows a configuration in which actuator 360 is attached to substrate 351 that has a lessened thickness near or at mid-length of actuator 360. In this configuration, maximum displacement may be achieved near or at mid-length of actuator 360. FIG. 3H-3 shows a configuration in which actuator 360 is attached to substrate 351 with a constraint imposed at the proximal end. In this configuration, maximum displacement may be achieved at the free distal end of actuator 360. Thus, desirable action of actuator 360 may be achieved modulating the flexibility of the substrate (e.g., by suitable selection of the width, thickness or other dimensions of the substrate).

In some embodiments, the opening to lumen 120 at the distal tip of an instrument may incorporate passive mechanical motion. FIGS. 31-1 and 31-3 illustrate incorporating a passive action in distal tip 300 of instrument 380, according to one embodiment of the present invention. As shown in FIG. 3I-1, at the opening of lumen 120, distal tip 300 may include diamond-patterned structure 390 formed out of braided wire (e.g., round or flat braided wire) or out of a net-shaped component (e.g., laser-cut tubing). (Laser-cut tubing has been used in stent construction, as known to a person of ordinary skill in the art.) In FIGS. 31-1 to 31-3, a polymeric covering layer may be provided over diamond-patterned structure 390, but this polymeric layer is omitted to allow diamond-patterned structure 390 to be clearly shown. Diamond-patterned structure 390 consists of rows of diamond shaped elements. The angle in each diamond element for rows at the distal end of diamond-patterned structure 390 decreases as one approaches the rows at its proximal end, such as shown in FIG. 3I-2. In this configuration, when a blood clot is engaged, such that the opening to lumen 120 of distal tip 300 is occluded, the suction pressure of the aspiration in lumen 120 drops. The drop in suction pressure compresses the tubular structure of distal tip 300 axially, which causes diamond-pattern structure 390 to expand radially outward increasingly from its proximal portion to its distal portion, thereby increasing the size of the opening to lumen 120, as shown in FIG. 3I-3.

FIGS. 3J-1 to 3J-4 illustrate incorporating a second passive mechanical action to distal tip 300, according to another embodiment of the present invention. FIG. 3J-1 shows distal tip 300 in a quiescent condition. Distal tip 300 includes tubular flexible region 395, which is delimited by rings 397-1 and 397-2 behind the opening to lumen 120. Rings 397-1 and 397-2 may be formed of material of a higher modulus than tubular flexible region 395. Multiple flexible cantilever beams oriented along the axial direction may be attached to both rings 397-1 and 397-2. (Although FIG. 3J-1 to 3J-4 each show only single cantilever beam 396, it is understood that cantilever beams such as cantilever beam 396 are provided around flexible region 395 at regular or variable angular intervals, each being attached to both rings 397-1 and 397-2.) Although rings 397-1 and 397-2 have relatively higher modulus than flexible region 395, they are also compliant enough to allow their individual inner radius to expand for passage of a blood clot, when the blood clot urges against the ring.

Initially, upon encountering a blood clot at the opening to lumen 120, the suction pressure inside lumen 120 drops, urging tubular flexible region 395 to compress radially and elongates axially, which urges tubular flexible region 395 to move towards the blood clot. The force urging forward motion of flexible region 395 is transmitted through the cantilever beams (e.g., cantilever beam 396) to flare out distal tip 300 at the opening to lumen 120. This condition is shown in both perspective and side view, respectively, in FIGS. 3J-2 and 3J-3. The enlarged flared-out opening to lumen 120 allows the blood clot to be ingested and pulled inward by the suction pressure. FIG. 3J-4 shows ingested blood clot 399 urging against ring 397-1, which enlarges the inner radius of ring 397-1, thereby increasing the chance that blood clot 399 may be pulled into tubular flexible region 395 by the suction pressure.

It is known to provide an inner lining to the surface of an aspiration conduit in an aspiration instrument. The conventional distal tip of an instrument is provided either slightly rounded, or beveled (i.e., forwardly angled or sloped). Such a distal tip is atraumatic to the surrounding tissue as the distal tip navigates through the blood vessel. A beveled tip may increase the cross-sectional area of the opening to the aspiration conduit into which the clot may be ingested. In such an aspiration instrument, the inner-most tubular layer (e.g., the walls of the aspiration conduit traversed by an ingested blood clot) may be, for example, an etched-PTFE layer, Pebax, or another material. Surrounding the inner layer or layers may be a coil or braided layer, with a urethane or Pebax material reflowed over the coil or braided layer to form a consistent structure. As an inner layer of etched-PTFE is not a melt-processable material and is therefore limited in geometry to the straight tube configuration. However, according to one embodiment of the present invention, the inner layer of an instrument may be provided by either Pebax (e.g., nylon) or PVDF, which is a melt-processable fluoropolymer. Both Pebax and PVDF may be used to create geometries at the preferred size of the liner suitable for removing clot.

According to one embodiment of the present invention, a patterned or textured liner (e.g., the surface of a layer of PVDF) may be provided along a section or the entire length of the aspiration conduit of the instrument, especially at the distal tip of the instrument. FIG. 4A-1 shows textured surface 410 provided on the walls of lumen 120 in distal tip 300 of an instrument. Such a liner may facilitate additional movements and may impart additional stress on the ingested blood clot or clot fragment, thereby further facilitating breaking the ingested clot or clot fragment down to even more digestible pieces. Such a liner may also cause movement in the ingested clot or clot fragment in a way that facilitates its ingestion. A health care professional may also enhance these actions by steering the instrument in a forward-and-backward vibratory axial motions, with or without concurrent vibration by the actuators at the distal tip of the instrument, and with or without providing suction pressure.

According to one embodiment of the present invention, one or more sensors or transducers may be provided in conjunction with the EAP actuator. For example, one or more portions of the patterned or textual layer (e.g., at the distal end) may serve as an electroactive sensor. The sensor may be located anywhere within the catheter body, or external to the catheter body (e.g., in a tubing attached to the catheter). The sensor may be used to detect the presence of a blood clot and to provide an electrical signal as sensory output. The sensory output of the electroactive sensor, or other sensors may be fed back to the controller to modulate pressure of the suction, the frequency or amplitude of an EAP actuator's vibration, or any combination thereof, so as to help with carrying out the thrombectomy.

FIGS. 4A-2 and 4A-3 show side and perspective views of exemplary textured surface 410, respectively, provided as steps cut in the inside wall of lumen 120.

FIGS. 4A-4 and 4A-5 show transverse cross-sectional views of textured surface 410, provided in the form of patterned stepped protrusions, as viewed into the opening of lumen 120 and as viewed out to the opening of lumen 120 from inside the distal tip, respectively.

FIGS. 4A-6 to 4A-8 show (a) side and perspective transverse cross-sectional views, and (b) a perspective view of textured surface 410, provided in the form of another patterned stepped protrusions.

According to another embodiment of the present invention, the inner layer may be etched to provide a “fluted” surface on the wall of the aspiration conduit. A fluted surface is one with raised structures that extends axially and that are separated from each other by gaps or channels that also extend axially. FIG. 4B shows the wall of lumen 120 having raised portions 411 separated by channels 412. Raised portions 411 may have a textured or patterned surface (e.g., formed using PVDF), or may have a smooth surface using a suitable material.

According to another embodiment of the present invention, the wall of the aspiration conduit is etched to provide etched depressions. For example, the etched depression may form a “rifled” channel (i.e., a continuous helical groove) running along a portion of the entire length of the distal tip or even along a substantial length or the entire length of the instrument. FIGS. 4C-1 and 4C-2 show a transverse cross-sectional view and a perspective view, respectively, of helical groove 422 on the enclosing walls of lumen 120. In other embodiments, the patterned surface may even be formed into a helical screw-like structure, such that, in conjunction with the motions of one or more actuators, an ingested clot rotates and presses against the inner patterned surface as it traverses through the instrument. The size of the ingested clot may be reduced due to attrition along the aspiration conduit. FIGS. 4D-1 and 4D-2 show a transverse cross-sectional view and a perspective view, respectively, of helical screw-like raised structure 423 on the enclosing wall of lumen 120, according to one embodiment of the present invention.

In other embodiments, the patterned surface may be in the form of scales (e.g., in configuration of familiar patterns akin to those provided on the bottom of a cross-country ski, snake skins, or a wood rasp).

The textured surface on the sidewalls of lumen 120 may incorporate an actuator or a movable or extendible element to promote preferential proximal migration of an ingested blood clot. FIGS. 5A-1 to 5A-3 illustrate the operation of textured structure 500 provided on the enclosing sidewalls of lumen 120, according to one embodiment of the present invention. As shown in FIG. 5A-1, structure 500 includes high modulus mobile element 501—which may be itself an EAP actuator—that can move or extend axially relative to stationary flexure beams 502, which are provided to constrain the lateral (i.e., radial) movements of high modulus mobile element 501. Textured structure 500 also includes a compliant region formed by stiffening flexure beams 504, which are connected laterally by elongating components 503. Elongating components 503 act as a compressible spring pushing against pressure from high modulus mobile element 501's axial motion towards the proximal portion of distal tip 300, while stretching the compliant region laterally. EAP actuator 506, which extends axially over the entire length of the compliant region, is attached to high modulus mobile element 501 at one end and, at the other end, to the proximal portion of distal tip 300 beyond the compliant region. In this embodiment, as actuator 506 is attached on the outside of the lining to lumen 120, it is not shown in FIG. 5A-1.

FIG. 5A-2 is a perspective view of distal tip 300 that has two copies of textured structure 500 provided on opposite sidewalls of lumen 120. For clarity of reference in the following description, actuator 506 is also not shown in FIG. 5A-2. FIG. 5A-3 shows actuator 506 provided over elongating components 503 in the compliant region of structure 500. When a blood clot is engaged and urged by aspiration suction pressure to move proximally inside lumen 120, corking may occur, which causes the suction pressure in lumen 120 to drop. Actuator 506 may be activated in a vibrational manner to cause high modulus mobile element 501 to engage in an axial forward-and-backward motion. At the same time, elongating components 503 in the compliant region are also engaged in motion. While providing a restoring force pushing back on high modulus mobile element 501 during actuator 506's relaxing phase, elongating components 503's lateral motion increases the diameter of lumen 120 during actuator 506's activated phase. This radial expansion of lumen 120 facilitates the ingested clot or clot fragment to move towards the proximal end of textured structure 500. Stiffening flexure beams 504 constrains bending of elongating components 503 to preserve structural integrity of distal tip 503. The combined effects of axial and radial motions of textured structure 500 can be very effective in uncorking lumen 120 and in facilitating the ingested blood clot or clot fragment to move in the preferential direction.

FIGS. 5B-1 to 5B-2 illustrate the operation of second textured structure 550 provided on the sidewalls of lumen 120, according to one embodiment of the present invention. Textured Structure 550 operates according substantially to the same principles as those of textured structure 500 of FIGS. 5A-1 to 5A-3. However, elongating components 503 in the compliant region is replaced by membrane 553, which is formed of a material having a lower modulus than high modulus mobile element 551. As shown in FIG. 5B-1, textured structure 550 includes higher modulus mobile element 551 that can move axially relative to stationary flexure beams 552 when EAP actuator 556 (not shown) is activated. Membrane 553 in the compliant region is attached at end 557a to high modulus mobile element 551 and at end 557b to a proximal portion of distal tip 300. EAP actuator 503 is affixed to textured structure 550 outside of lumen 120, as shown in FIG. 5B-2. Membrane 553 is connected to stiffening flexure beams 554, which constrain membrane 553's movements to within its plane, when actuator 556 is activated. When activated, EAP actuator 556 causes high modulus mobile element 551 to move towards the proximal end of textured structure 550 and stretches the region of low modulus material laterally, which increases the size of lumen 120. Stiffening flexure beams 554 constrains bending the region of lower modulus material to preserve structural integrity. Thus, the motions of high modulus mobile element 551 and membrane 553 are substantially similar to those described above with respect to their counterparts in FIGS. 5A-1 to 5A-3. In some embodiments, EAP actuator 553 may also be embedded in membrane 553, which may be formed in this instance by two laminate layers of the lower modulus material. As shown in FIG. 5B-2, two copies of textured structure 550 may be provided on opposite sidewalls of lumen 120. The combined motions of high modulus mobile element 551 and membrane 553 are effective in uncorking lumen 120 and in urging the ingested blood clot or clot fragment to move preferentially towards the proximal end of distal tip 300.

Various additional embodiments are designed to improve flexibility in the funnel-shaped opening to lumen 120 when in the flared open position, while preserving the opening to a fixed size in the collapsed position. FIGS. 6A-1 and 6A-2 illustrate clamshell structure 610 for controlling the opening to lumen 120, according to one embodiment of the present invention; clamshell structure 610 has a soft-opening and a stiff closure limit. As shown in FIG. 6A-1, clamshell structure 600 at distal tip 300 of the instrument includes first and second structural halves 600a and 600b connected by elastic elements 601a and 601b. A compliant polymer covering—not shown for clarity in the following description-is provided over clamshell structure 610. First and second structural halves 600a and 600b are hinged at planar hinge flexures 602a and 602b, which are integrally formed on the proximal portion of distal tip 300. Planar hinge flexure 602b is provided at 180 degrees on the opposite side of distal tip 300. FIG. 6A-1 shows clamshell structure 610 at its collapsed position, maintaining a strict closure limit for the opening of lumen 120. Elastic elements 601a and 601b may each be operated by an EAP actuator (not shown) that stretches the polymer covering to cause first and second structural halves 600a and 600b to flare out, as shown in FIG. 6A-2. In some embodiments, the tension in the polymer covering may be sufficient to cause elastic elements 601a and 601b sufficiently to place clamshell structure 610 to its open position. This configuration provides a large displacement at the opening to lumen 120, but provides a strict closure limit of the opening at clamshell structure collapsed position.

FIGS. 6B-1 and 6B2 illustrate stent-like mechanical structure 650 for controlling the opening to lumen 120, according to one embodiment of the present invention; like clamshell structure 610 of FIGS. 6A-1 and 6A-2, stent-like mechanical structure 650 also allows a soft opening, but preserves a stiff closure limit, for the opening to lumen 120. As shown in FIG. 6B-1, stent-like mechanical structure 650 is provided over compliant (i.e., low modulus) polymer layer 651, which forms the inner walls of lumen 120. Stent-like mechanical structure 650, which may be a connected laser-cut mechanical framework formed out of NiTi or any other suitable material. FIG. 6B-1 shows the relaxed or collapsed condition of stent-like mechanical structure 650. FIG. 6B-2 shows the opened condition of compliant structure 650. Thus, stent-like mechanical structures 650 has the advantage of low stiffness during its opening, while having the high stiffness during its collapsed position to maintain a strict closure limit which preserves the opening to the aspiration conduit from collapsing.

Alternatively, a compliant structure for controlling the opening to lumen 120 may also be formed using a foldable membrane. FIGS. 6C-1 to 6C-3 illustrate the operation of tubular compliant structure 680 for controlling lumen 120 using a foldable membrane material, according to one embodiment of the present invention. FIG. 6C-1 shows distal tip 300 having tubular compliant structure 680 provided at the opening to lumen 120 of distal tip 300. A polymeric covering (not shown) may be provided over tubular compliant structure 680. Tubular compliant structure 680 is attached to non-compliant base 681 at proximal portion 681 of distal tip 300. Tubular compliant structure 680 includes numerous stiff sliding plates 693 connected to each other by sheets 692 of the foldable membranes. As shown in FIG. 6C-1, tubular compliant structure 680 is in its collapsed or closed position. FIG. 6C-2 shows tubular compliant structure 680 is in its open position. FIG. 6C-3 shows the configurations of tubular compliant structure 680 in its open position (A) and in its closed or collapsed positions (B). In position A, stretching of the polymeric covering (e.g., by EAP actuators) causes foldable membrane sheets 692 to become slack and sliding plates 693 to be spread out at their opening positions (i.e., with minimum overlap), thereby providing a large displacement from tubular compliant structure 680's collapsed position. At position B, however, foldable membrane sheets 692 are taut, which causes sliding plates 593 to be at their maximum overlaps, thus preserving a minimal diameter at the opening to lumen 120.

FIG. 7A shows integrated thrombectomy device or instrument 700, in accordance with one embodiment of the present invention. As shown in FIG. 7A, integrated thrombectomy device 700 includes distal tip 724, flexible catheter body 726 and digital controller 728. Distal tip 724 includes integrated EAP actuator 725 and dilator 721. As mentioned above, the term “integrated EAP actuator” refers collectively to one or more EAP actuators, together with other electrical and mechanical elements, to form a component for building a functional device. Dilator 721 is shown to have a tapered profile. Flexible catheter body 726 provides a large-bore lumen that runs continuously throughout its length to provide suction and to serve as a fluid conduit. In one embodiment, the lumen at distal end 722 of flexible catheter body 726 may be provided a larger lumen than the rest of flexible catheter body 726 to enhance suction pressure at the distal end. The proximal end of flexible catheter body 726 includes port 727, which is adapted for connection to an aspiration device (not shown) to provide the aspiration suction pressure and to allow for fluid disposal. Integrated EAP actuator 725 receives electrical signals from digital controller 728 over electrical conductors or traces that are embedded in the cylindrical wall along the entire length of flexible catheter body 726, such as shown in inset 723. In one embodiment, the electrical traces include at least two copper coils and at least one stainless steel coil. The copper coils are provided to carry electrical control and data signals between digital controller 728 and integrated EAP actuator 725. The stainless-steel coil provides structural strength to flexible catheter body 726 to prevent the large-bore lumen therein from collapsing under aspiration suction pressure (i.e., at an aspiration pressure below 30 in. Hg) and to prevent kinks from forming along flexible catheter body 726. If desired, additional supportive rings or bands (e.g., metal rings, such as stainless-steel rings) may be provided at suitable locations along flexible catheter body 726 (e.g., in the proximity of EAP actuator 725) to constrain inward or longitudinal movements of EAP actuator 725 and to provide additional structure strength and integrity to flexible catheter body 726.

At distal tip 724, one or more radiopaque markers may be provided to guide navigating distal tip 724 through the vasculature. As shown in FIG. 7A, a radiopaque marker is provided between integrated EAP actuator 725 and dilator 721. Dilator 721 is shaped to facilitate distal tip 724 thread through the vasculature to its destination. Dilator 721 may be configured to expose or to open up the large-bore lumen for clot ingestion, using any of the techniques described above.

FIG. 7B is an enlarged view of integrated EAP actuator 725 at distal tip 724 of integrated thrombectomy device 700, in accordance with one embodiment of the present invention. FIG. 7B shows integrated EAP actuator 725 at a later step of its formation. As shown in FIG. 7B, integrated EAP actuator 725 may be formed circumferentially on the outer surface of mandrel 702. At the end of integrated EAP actuator 725's formation, mandrel 702 is removed to provide a large-bore lumen. Mandrel 702 may be, for example, a PTFE glass-filled cylinder or tube with an outer circumference that matches that of the desirable large-bore lumen in integrated EAP actuator 725. First to form on mandrel 702 is inner polymeric layer 703 of integrated EAP actuator 725. Inner polymeric layer 703 may be a Pebax layer. In some embodiments, multiple layers of Pebax of varying durometers may be provided for transition.

Provided on the outer surface of inner polymeric layer 702 is polyimide flexible circuit 704. Polyimide flexible circuit 704 may have provided thereon (e.g., using conductive ink) two or more electrodes 707 for attaching EAP actuators. As shown in FIG. 7B, EAP actuator 705 is formed over polyimide flexible circuit 704. EAP actuator 705 is shown electrically contacted, for example, at location 706. EAP actuator 705 may be formed out of one or more suitable electroactive polymers described above. In one embodiment, EAP actuator 705 is formed from a rectangular sheet (e.g., 11.0 mm by 23.5 mm) and then rolled circumferentially around inner polymeric layer 720, such that the opposite sides of the longer dimension are abutting. Electrodes 707 may be electrically connected to the copper coils embedded in flexible catheter body 726 using a wire soldering technique, for example. In one embodiment, two copper coils and one stainless steel coils are embedded in flexible catheter body 726, each coil provided at 26 wraps per inch, for example. In one embodiment, as shown in FIG. 7B, outer jacket 710 of Pebax (with varying durometers for transitions, if desired) is provided along the outer surface of flexible catheter body 726, as far distally up to or close to polyimide flexible circuit 704 of integrated EAP actuator 725. The remainder of integrated EAP actuator 725, indicated in FIG. 7B by ovoid 701, is encapsulated in a Tecoflex material, using a technique described below in conjunction with FIG. 7C. The Tecoflex material may be obtained, for example, as an adhesive based on a fast-crystallizing polyurethane resin.

FIG. 7C illustrates formation of the Tecoflex encapsulation in integrated EAP actuator 725, in accordance with one embodiment of the present invention. A short sheath section of Tecoflex is provided over integrated EAP actuator 726, with its end overlapping mandrel 702 and outer jacket 710. In one embodiment, the Tecoflex sheath may be, for example, 0.003″ thick. As shown in FIG. 7C, Tecoflex sheath 732 has proximal end 731 overlapping outer jacket 710 and distal end 733 overlapping mandrel 702. Heat is then applied to proximal end 731 and distal end 733 to their polymeric materials with the inner polymeric layer 703 and the outer jacket 710 (i.e., the Tecoflex melts and reflows into the hydrophilic coating at distal end 733 and the Pebax at proximal end 731). In this manner, integrated EAP actuator 725 is hermetically encapsulated without having Tecoflex sheath 732 blended into EAP actuator 705.

A hydrophilic coating may be applied to integrated thrombectomy device 700 to enhance navigation properties and outer layer lubricity.

The above detailed description is provided to illustrate specific embodiments of the present invention and is not intended to be limiting. Numerous modifications and variations within the scope of the invention are possible. The present invention is set forth in the accompanying claims below.

1. An instrument suitable for use in a medical procedure, the instrument being electrically connected to a controller that provides electrical control signals and mechanical suction, comprising:
a body having a distal end and a proximal end, the body having a conduit between the distal end and the proximal end through which a fluid moves under the suction; and
one or more actuators attached or embedded in the body, each actuator comprising one or more electroactive polymer layers that provides a mechanical motion at the distal end in response to stimulation by the electrical control signals, wherein the electrical control signals are provided according to at least one predetermined pattern.

2. The instrument of claim 1, wherein the predetermined pattern enables two or more groups of the actuators to be activated sequentially.

3. The instrument of claim 1, wherein the predetermined pattern enables two or more groups of the actuators to be activated concurrently.

4. The instrument of claim 1, wherein the predetermined pattern enables two or more groups of the actuators to be activated sequentially, one group after another, and wherein, within each group, two or more actuators are actuated concurrently.

5. The instrument of claim 4, wherein the actuators each extend along a longitudinal direction along a length of the body.

6. The instrument of claim 5, wherein the two or more groups of actuators are each located at a radial distance from a longitudinal axis of the conduit.

7. The instrument of claim 1, wherein the mechanical suction varies in a coordinated fashion with the predetermined pattern.

8. The instrument of claim 7, wherein a pressure of the mechanical suction is decreased when one of the mechanical motions comprise a longitudinal motion at the distal end of the body.

9. The instrument of claim 1, wherein the distal end of the body has an opening that exposes the conduit, and wherein a portion of the body at the opening has a funnel-like shape.

10. The instrument of claim 9, wherein the actuators are provided behind the opening.

11. The instrument of claim 1, wherein a first one of the actuators wraps around the body helically.

12. The instrument of claim 11, wherein a second one of the actuators also wraps around the body helically but in a different chirality than the first actuator.

13. The instrument of claim 1, wherein a first one of the actuators wraps substantially circumferential around the body relative to a longitudinal axis of the body.

14. The instrument of claim 13, wherein the first actuator has a first end and a second end, and wherein the first end and the second end abut each other during that actuator's mechanical motion.

15. The instrument of claim 13, wherein the first actuator has a first end and a second end, wherein the first end and the second end are separated from each other by a gap, and wherein the actuator attaches to a stiffening material that spans the gap.

16. The instrument of claim 1, further comprising a flexible circuit on which the actuators are mounted.

17. The instrument of claim 1, further comprising a mass placed on one of the actuators to modify a resonant frequency of the mechanical motion of that actuator.

18. The instrument of claim 17, wherein the mass comprises a radiopaque material.

19. The instrument of claim 17, wherein the mass comprises tungsten.

20. The instrument of claim 1, wherein each actuator comprises a first end and a second end, wherein one or both ends of the actuator are stiffened to constrain the mechanical motion of that actuator.

21. The instrument of claim 1, wherein each actuator is embedded or attached to a portion of the body that comprises a high-modulus material, the high-modulus material having a modulus that is higher than that the actuator's electroactive polymer layers, the portion of the body serving as a substrate.

22. The instrument of claim 21, wherein the portion of the high-modulus material is positioned to guide the mechanical motion.

23. The instrument of claim 21, further comprising (i) a flexible circuit formed on the substrate, and (ii) electrodes provided on the flexible circuit to provide the electrical signals to the actuators.

24. The instrument of claim 1, wherein each actuator is embedded or attached to a portion of the body that has a low-modulus material with a modulus lower than those of the electroactive polymer layers, the portion of the body serving as a substrate.

25. The instrument of claim 1, wherein the body comprises a patterned or textured layer of material exposed to the conduit.

26. The instrument of claim 25, wherein the patterned or textured layer of material comprises one or more of: polyvinylidene fluoride (PVDF), trifluoroethylene (TrFE), 1,1-chlorofluoroethylene (CFE), chlorotrifluoroethylene (CTFE).

27. The instrument of claim 25, wherein the patterned or textured layer of material comprises a copolymer of polyvinylidene fluoride (PVDF) and one or more monomers selected from the group consisting of trifluoroethylene (TrFE), 1,1-chlorofluoroethylene (CFE), chlorotrifluoroethylene (CTFE).

28. The instrument of claim 25, wherein the patterned or textured layer has one or more fluted or raised portions, wherein adjacent fluted or raised portions are separated from each other by a channel.

29. The instrument of claim 28, wherein each raised portion further includes a smooth surface.

30. The instrument of claim 28, wherein each raised portion further includes a textured surface.

31. The instrument of claim 25, wherein the patterned or textured layer includes etched depressions.

32. The instrument of claim 31, wherein one of etched depressions is provided in as a rifled helical pattern extending longitudinally along the body.

33. The instrument of claim 25, wherein the patterned or textured layer is formed in a helical screw pattern that extends longitudinally along the body.

34. The instrument of claim 25, wherein the patterned or textured layer incorporates a mobile element capable of axial motion.

35. The instrument of claim 34, further including flexure beams that constrain the mobile element in primarily axial motion.

36. The instrument of claim 35, wherein the patterned or textured layer further comprises a compliant region that has greater compliant than a region within which the mobile moves.

37. The instrument of claim 36, wherein the compliant region comprises flexure beams that are interconnected by elongation elements.

38. The instrument of claim 37, wherein the elongation elements urge against the flexure beams of the compliant region resulting in a radial expansion of the conduit.

39. The instrument of claim 1, further comprising a mechanical structure that opens to the conduit, the mechanical structure enabling a flared opening to the conduit in one of two positions, while the mechanical structure preserves a closure limit to a size of the conduit in the other one of the two positions.

40. The instrument of claim 39, wherein the mechanical structure is covered externally by a polymer covering.

41. The instrument of claim 39, wherein the mechanical structure comprises a first portion and a second portion attached to each other by one or more elastic elements.

42. The instrument of claim 39, wherein the mechanical structure comprises a plurality of movable plates connected to each other by sheets of a foldable membrane material.

43. The instrument of claim 1, further comprising one or more radiopaque elements provided at the distal end of the body.

44. The instrument of claim 1, wherein a hydrophilic coating is provided at the distal end of the body.

45. The instrument of claim 1, further comprising one or more supportive bands on the body to provide mechanical support.

46. The instrument of claim 45, wherein the supportive bands each comprise stainless steel.

47. The instrument of claim 45, wherein the supportive bands maintain the conduit open when a pressure of the suction is at or below 30 inHg.

48. The instrument of claim 1, further comprising one or more supportive bands on the body to constrain longitudinal or axial motion of one or more of the actuators.

49. A device suitable for use in a medical procedure, the device being configured to operate in accordance with electrical signals received from a controller, the device comprising a catheter body having a proximal end and a distal end, the distal end comprising a tapered tip and an integrated electroactive polymer (EAP) actuator provided between the tapered tip and the proximal end of the body, wherein (i) the catheter body includes a lumen which forms a fluid conduit running continuously throughout the catheter body's length to allow fluid to be received from an opening at the tapered tip and to flow to the proximate end of the catheter body under suction pressure, (ii) the catheter body further comprises two or more electrical conductors in the catheter body to carry the electrical signals between the controller and the integrated EAP actuator, (iii) the EAP actuator is stimulated by the electrical signals into motion, and (iv) the electrical signals and the suction pressure are varied in a coordinated fashion according to one or more predetermined patterns.

50. The device of claim 49, wherein the tapered tip comprises a dilator.

51. The device of claim 49, wherein the conduit has a greater radius at the distal end of the catheter body than at the proximal end of the catheter body.

52. The device of claim 49, further comprising a port in the proximal end of the catheter body, the port being opened to the conduit and being adapted for connection to an aspiration device.

53. The device of claim 52, wherein the electrical conductors are each helically embedded in the catheter body.

54. The device of claim 49, further comprising one or more wires helically embedded in the catheter body to provide mechanical support.

55. The device of claim 54, wherein the wires are formed out of stainless steel.

56. The device of claim 49, wherein the conduit is provided mechanical support in the catheter body to remain open at a pressure equal to or below 30 in. Hg.

57. The device of claim 49, further comprising one or more supportive rings or bands to provide mechanical support.

58. The device of claim 57, wherein supportive rings or bands constrain inward or longitudinal movements of EAP actuator.

59. The device of claim 57, wherein one or more of the supportive rings or bands serve as a radiopaque marker for guiding navigation of the distal end of the catheter body through vasculature.

60. The device of claim 49, wherein the integrated EAP actuator comprises one or more EAP actuators and an inner polymeric layer.

61. The device of claim 60, wherein the inner polymeric layer comprises Pebax.

62. The device of claim 60, wherein the inner polymeric layer comprises multiple layers of different durometers.

63. The device of claim 60, wherein the integrated EAP actuator further comprises a flexible circuit having electrodes for attaching and electrically connecting the EAP actuators.

64. The device of claim 63, wherein the flexible circuit further comprises conductive ink, provided for electrically connecting the conductors in the catheter body to the electrodes.

65. The device of claim 60, wherein the EAP actuators are each formed from a rectangular sheet rolled circumferentially around the inner polymeric layer, such that opposite sides of a dimension of the rectangle are abutting.

66. The device of claim 60, wherein the conductors in the catheter body are each embedded in the catheter body in the form of a conductive coil.

67. The device of claim 60, further comprising an outer jacket provided along an outer surface of the catheter body except at the integrated EAP actuator.

68. The device of claim 67, wherein the integrated EAP actuator is encapsulated in a thermoplastic polyurethane (TPU) material.

69. The device of claim 68, wherein a portion of the distal end of the catheter body is provided a hydrophilic coating.

70. The device of claim 69, wherein the TPU material attaches to the inner polymeric layer and the outer jacket.
